(19) United States
(12) Patent Application Publication
(10) Pub. No.: US 2022/0022771 A1
(43) Pub. Date: Jan. 27, 2022

(54) ELECTRICAL IMPEDANCE SPECTROSCOPY FOR EVALUATION OF EXCISION-REQUIRED SUSPICIOUS MASSES

(71) Applicants: Mohammad Abdolahad, Tehran (IR); Reihane Mahdavi, Tehran (IR); Sajad Mehrvarz, Tehran (IR); Narges Yousefpour, Tehran (IR); Hossein Ataee, Tehran (IR); Saeed Abdolhosseini, Tehran (IR); Naser Namdar Habashi, Tehran (IR); Farzaneh Hajighasemi, Tehran (IR); Hadi Ghafari, Tehran (IR)

(72) Inventors: Mohammad Abdolahad, Tehran (IR); Reihane Mahdavi, Tehran (IR); Sajad Mehrvarz, Tehran (IR); Narges Yousefpour, Tehran (IR); Hossein Ataee, Tehran (IR); Saeed Abdolhosseini, Tehran (IR); Naser Namdar Habashi, Tehran (IR); Farzaneh Hajighasemi, Tehran (IR); Hadi Ghafari, Tehran (IR)

(73) Assignee: Nano Hesgarsazan Salamat Arya, Tehran (IR)

(21) Appl. No.: 17/492,651

(22) Filed: Oct. 3, 2021

Related U.S. Application Data
(60) Provisional application No. 63/105,213, filed on Oct. 24, 2020.
(60) Provisional application No. 63/087,183, filed on Oct. 3, 2020.

(51) Int. Cl. A61B 5/06; A61B 5/00; A61B 5/25; G01N 33/574

(52) U.S. Cl. CPC A61B 5/063; A61B 5/0075; G01N 33/57415; A61B 5/25; A61B 5/6848; A61B 5/4312

(57) ABSTRACT

A method for real-time and in-vivo detecting cancerous status of a suspected mass to in a living body. The method includes putting two electrodes of an electrical probe in contact with the suspected mass, recording an electrical impedance spectroscopy (EIS) from the suspected mass utilizing an impedance analyzer device connected to the electrical probe by plotting an impedance phase diagram respective to a swept range of frequencies while applying an alternating current (AC) voltage between the two electrodes, calculating an impedance phase slope (IPS) of the plotted impedance phase diagram in a frequency range between 100 kHz and 500 kHz, and detecting cancerous status of the suspected mass based on the calculated IPS. Detecting cancerous status of the suspected mass based on the calculated IPS includes detecting the suspected mass is a cancerous mass or a precancerous mass if the calculated IPS is less than a reference IPS.

### CROSS-REFERENCE TO RELATED APPLICATION

This application claims the benefit of priority from pending U.S. Provisional Patent Application Ser. No. 63/087,183 filed on Oct. 3, 2020, and entitled “BIOELECTRICAL PATHOLOGY OF THE BREAST” and pending U.S. Provisional Patent Application Ser. No. 63/105,213 filed on Oct. 24, 2020, and entitled “BIOPSY-FREE CANCER DIAGNOSTIC NEEDLE FOR REAL-TIME DISTINGUISHMENT OF BENIGN AND MALIGNANT BREAST MASSES WITH BI-RADS AND PATHOLOGICAL CALIBRATIONS”, which are both incorporated herein by reference in their entirety.

### TECHNICAL FIELD

The present disclosure generally relates to diagnosis of excision-required cancerous or precancerous masses, and particularly, to an electrical probe, a system, and a method for diagnosis of an excision-required cancerous or precancerous mass by analyzing electrical mass spectroscopy of a suspicious mass.

### BACKGROUND

Lesions of uncertain malignant potential (histologically classified as breast imaging-reporting and data system3 (BI-RADS3)) have become a significant health issue today. Classifying a lesion as probably benign (BI-RADS3) in sonography means that about 2% or less chance of malignancy exists in a lesion. A short-term (6 months) follow-up is recommended of BI-RADS3 findings that unfortunately are sometimes ignored by patients and it may lead to a late diagnosis of probably high-risk lesions, for example, borderline breast lesions. Borderline breast lesions mostly occur in younger patients with dense breast masses with a higher percentage of glandular tissue that is challenging in mammographic evaluations with a high rate of false-negative results. Although only about 2.5% of breast cancer cases occur in women under 35, it is the most prevalent cause of mortality in 15 to 34 years old patients. As several studies reported a higher prevalence of adverse prognostic factors in young women with breast cancer, precise and early detection of high-risk lesions is especially necessary for young patients with a family history of breast cancer and young women of gestational age planning to become pregnant.

Also, malignant probability of breast lesions scored as BI-RADS4 (from BI-RADS4a. to BIRADS4c) is in a considerable span of 2-95% (BI-RADS4a: 2-10%, BI-RADS4b: 10-50%, BI-RADS4c: 50-95%). Therefore, a differential diagnosis between benign, high-risk, and malignant breast masses scored as BI-RADS4, mostly 4a, has become extremely important.

On the other hand, few numbers of core needle biopsy (CNB) from just specific regions in large diameter or heterogeneous tumors may miss some critical lesions that must be evaluated by a pathologist. Examples of these lesions include fibroepithelial lesions (FEL) with cellular stroma and phyllodes tumors (PT), spindle cell lesions, papillary lesions, mucinous lesions, radial scar, atypical proliferative lesions including flat epithelial atypia (FEA), atypical ductal hyperplasia (ADH), atypical lobular hyperplasia (ALH), and microcalcifications not associated with a specific pathology but suspicious to be the origin of ductal carcinoma in-situ (DCIS). Therefore, achieving the most pathologic portions of these lesions is valuable in diagnosing and possible treatments.

Therefore, a correct discrimination between BI-RADS3 and BI-RADS4a lesions for taking biopsies and precise selection in a tumor region for an accurate biopsy is complicated for radiologists while it is vital for patients to prevent misdiagnose of high-risk or borderline breast masses. Nowadays, such diagnoses are dependent on expert and skill of radiologists. There are some advanced techniques to increase biopsy accuracy, such as stereotactic biopsy, ultrasound elastography, multi-parametric MRI/TRUS fusion biopsy, contrast-enhanced MRI, and high-velocity pulse biopsy, which are primarily complicated and expensive.

Hence, there is a need for a highly precise, simple, and real-time tumor detection system and method that is capable of focusing on detecting borderline breast masses or high-risk benign lesions that are highly recommended to be excised by pathological guidelines. In fact, there is a need for detecting lesions that may be missed as BIRADS3 in a sonographic evaluation, so no biopsy would be recommended for them.

### SUMMARY

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes a method for real-time and in-vivo detecting cancerous status of a suspected mass to be cancerous/precancerous in a living body. The method may include putting two electrodes of an electrical probe in contact with the suspected mass, recording an electrical impedance spectroscopy (EIS) from the suspected mass utilizing an impedance analyzer device connected to the two electrodes of the electrical probe, calculating an impedance phase slope (IPS) of an impedance phase diagram in a frequency range between 100 kHz and 500 kHz utilizing one or more processors, and detecting cancerous status of the suspected mass based on the calculated IPS utilizing one or more processors.

In an exemplary implementation, recording the EIS from the suspected mass may include applying an alternating current (AC) voltage in a sweeping range of frequencies to the two electrodes of the electrical probe, measuring an electrical impedance magnitude of the suspected mass at a frequency of 1 kHz (Z_{1 kHz}), and plotting the impedance phase diagram by measuring a set of electrical impedance phase values from the suspected mass respective to the swept range of frequencies. In an exemplary implementation, calculating the IPS may include calculating an IPS of the plotted impedance phase diagram in a frequency range between 100 kHz and 500 kHz if the measured Z_{1 kHz} is less than a reference impedance value.

In an exemplary implementation, detecting cancerous status of the suspected mass based on the calculated IPS may include detecting the suspected mass being at least one of a cancerous mass and a precancerous mass if the calculated IPS is less than a reference IPS. In an exemplary implementation, detecting the cancerous status of the suspected mass may include detecting the suspected mass being a benign region if the calculated IPS is more than the reference IPS.

In an exemplary implementation, calculating the IPS of the plotted impedance phase diagram in the frequency range between 100 kHz and 500 kHz may include calculating the IPS from a relation defined by:

$$IPS = \frac{{Phase}_{2} - {Phase}_{1}}{log ( {Frequency}_{2} ) - log ( {Frequency}_{1} )} ,$$

Where, Phase₁ is a first measured impedance phase value at a first frequency value (Frequency₁) of 100 kHz and Phase₂ is a second measured impedance phase value at a second frequency value (Frequency₂) of 500 kHz.

In an exemplary implementation, exemplary method may further include generating a calibration dataset. In an exemplary implementation, generating the calibration dataset may include generating the reference impedance value and generating the reference IPS.

In an exemplary implementation, generating the reference impedance value may include measuring a set of Z_{1 kHz} values from a plurality of masses associated with a respective plurality of persons and determining the reference impedance value equal to a maximum value of the measured set of Z_{1 kHz} values.

In an exemplary implementation, generating the reference IPS may include calculating, utilizing one or more processors, a set of IPS values from the plurality of masses, determining cancerous status of each mass of the plurality of masses by applying a pathological assay to each mass, assigning the determined status of each mass to the respective calculated IPS value, classifying the set of IPS values into three IPS ranges based on the determined cancerous status of each mass of the plurality of masses, and determining the reference IPS based on the classified set of IPS values.

In an exemplary embodiment, the determined cancerous status may include one of a benign state, a cancerous state, and a precancerous state, based on result of the applied pathological assay. In an exemplary implementation, classifying the set of IPS values into three IPS ranges may include classifying the set of IPS values into one of a benign IPS range, a precancerous IPS range, and a cancerous IPS range. In an exemplary embodiment, the benign IPS range may include a first range of IPS values assigned as being of the benign state. In an exemplary embodiment, the precancerous IPS range may include a second range of IPS values assigned as being of the precancerous state. In an exemplary embodiment, the cancerous IPS range may include a third range of IPS values assigned as being of the cancerous state. In an exemplary implementation, determining the reference IPS based on the classified set of IPS values may include determining the reference IPS equal to a value between a minimum IPS value among IPS values of the benign IPS range and a maximum IPS value among IPS values of both the precancerous IPS range and the cancerous IPS range.

In an exemplary implementation, putting two electrodes of the electrical probe in contact with the suspected mass may include putting two electrodes of the electrical probe in contact with a suspected mass to be cancerous or precancerous that may be located in a breast tissue. In an exemplary embodiment, the reference impedance value may include an impedance value of 1500Ω and the reference IPS may include an IPS value equal to zero. In an exemplary implementation, detecting cancerous status of the suspected mass may include detecting the suspected mass being at least one of a cancerous mass and a precancerous mass in the breast tissue if the measured Z_{1 kHz} is less than a reference impedance value of 1500Ω and the calculated IPS is less than a reference IPS value of zero. In an exemplary implementation, detecting the cancerous status of the suspected mass may include detecting the suspected mass being a cancerous (malignant) breast lesion if the calculated IPS is less than an IPS value of −2 and detecting the suspected mass is a precancerous (borderline or high-risk) breast lesion if the calculated IPS is in a range of IPS value between −2 and 0. In an exemplary implementation, the method may further include detecting the suspected mass is a non-excision-required mass in the breast tissue including at least one of a fat necrotic region, a benign glandular region, and combinations thereof if the measured Z_{1 kHz} is more than the reference impedance value of 1500Ω.

In an exemplary implementation, putting two electrodes of the electrical probe in contact with the suspected mass may include putting two electrodes of the electrical probe in contact with a suspected nodule to be cancerous or precancerous that may be located in a thyroid gland. In an exemplary implementation, the reference impedance value may include an impedance value of 2000Ω, and the reference IPS may include an IPS value of 2. In an exemplary implementation, detecting the cancerous status of the suspected mass may include detecting the suspected nodule is one of a cancerous thyroid nodule and a precancerous thyroid nodule if the calculated IPS is less than an IPS value of 2 and the measured Z_{1 kHz} is less than 2000Ω.

In an exemplary implementation, applying the AC voltage in the sweeping range of frequencies to the two electrodes of the electrical probe may include connecting a first proximal end of a first electrode and a second proximal end of a second electrode of the electrical probe to the impedance analyzer device and applying an AC voltage in a sweeping range of frequencies between 1 kHz and 500 kHz to the two electrodes utilizing the impedance analyzer device.

In an exemplary implementation, putting the two electrodes of the electrical probe in contact with the suspected mass may include putting a first distal end of a first electrode and a second distal end of a second electrode of the electrical probe in contact with the suspected mass by inserting the first distal end of the first electrode and the second distal end of the second electrode inside the suspected mass.

In an exemplary implementation, inserting the first distal end of the first electrode and the second distal end of the second electrode of the electrical probe inside the suspected mass may include inserting a first distal end portion of the first electrode of the electrical probe through skin into the suspected mass and pushing a second distal end portion of the second electrode of the electrical probe through the first electrode into the suspected mass. Where, the first electrode may include a first electrically conductive needle including a hollow needle and the second electrode may include a second electrically conductive needle placed inside the first electrode.

In an exemplary embodiment, the first electrically conductive needle may include a stainless steel needle of at least one of a peripheral venous catheter and an intravenous (IV) cannula with a gauge size of 14 or more. In an exemplary embodiment, the second electrically conductive needle may include a stainless steel needle of a spinal cannula with a gauge size of 20 or more.

In an exemplary implementation, the method may further include preparing the electrical probe. In an exemplary implementation, preparing the electrical probe may include coating a second electrically insulating layer on the second electrically conductive needle except a distal end portion of the second electrically conductive needle, placing the second electrically conductive needle inside the first electrically conductive needle, covering an outer surface of the first electrically conductive needle with a first electrically insulating layer except a distal end portion of the first electrically conductive needle, and attaching two electrical connectors to the first electrically conductive needle and the second electrically conductive needle. In an exemplary implementation, attaching two electrical connectors to the first electrically conductive needle and the second electrically conductive needle may include attaching a first electrical connector onto a surface of the first electrically conductive needle adjacent to a proximal end of the first electrically conductive needle and attaching a second electrical connector onto a proximal end of the second electrically conductive needle.

In another general aspect, the present disclosure describes a system for real-time and in-vivo detecting cancerous status of a suspected mass to be cancerous or precancerous in a living body. The system may include an electrical probe, an impedance analyzer device configured to be connected to the electrical probe, and a processing unit electrically connected to the impedance analyzer device.

In an exemplary embodiment, the electrical probe may include a first electrode, a second electrode, a first electrically insulating layer, and a second electrically insulating layer. In an exemplary embodiment, the first electrode may include a first electrically conductive needle. In an exemplary embodiment, the first electrode may include a first distal end portion and a first proximal end portion. In an exemplary embodiment, the first electrically insulating layer may be placed around the first electrode except the first distal end portion.

In an exemplary embodiment, the second electrode may include a second electrically conductive needle. In an exemplary embodiment, the second electrode may be placed inside the first electrode. In an exemplary embodiment, the second electrode may be movable in longitudinal direction along the first electrode. In an exemplary embodiment, the second electrode may include a second distal end portion and a second proximal end portion. In an exemplary embodiment, the second distal end portion may be configured to be placed outside of the first electrode. In an exemplary embodiment, the second electrically insulating layer may be placed between the first electrode and the second electrode. In an exemplary embodiment, the first distal end portion and the second distal end portion may be configured to be put in contact with the suspected mass.

In an exemplary embodiment, the processing unit may include a memory having processor-readable instructions stored therein and a processor. In an exemplary embodiment, the processor may be configured to access the memory and execute the processor-readable instructions. In an exemplary embodiment, the processor may be configured to perform a method by executing the processor-readable instructions. In an exemplary embodiment, the method may include applying, utilizing the impedance analyzer device, an alternating current (AC) voltage in a sweeping range of frequencies to the first electrode and the second of the electrical probe, measuring, utilizing the impedance analyzer device, an electrical impedance magnitude of the suspected mass at a frequency of 1 kHz (Z_{1 kHz}), plotting, utilizing the impedance analyzer device, an impedance phase diagram by measuring a set of electrical impedance phase values from the suspected mass respective to the swept range of frequencies, calculating an impedance phase slope (IPS) of the plotted impedance phase diagram in a frequency range between 100 kHz and 500 kHz if the measured Z_{1 kHz} is less than a reference impedance value, and detecting cancerous status of the suspected mass based on the calculated IPS.

In an exemplary implementation, detecting the cancerous status of the suspected mass based on the calculated IPS may include detecting the suspected mass being at least one of a cancerous mass and a precancerous mass if the calculated IPS is less than a reference IPS, or detecting the suspected mass being a benign region if the calculated IPS is more than the reference IPS.

In an exemplary implementation, calculating the IPS of the plotted impedance phase diagram in the frequency range between 100 kHz and 500 kHz may include calculating the IPS from a relation defined by:

$$IPS = \frac{{Phase}_{2} - {Phase}_{1}}{log ( {Frequency}_{2} ) - log ( {Frequency}_{1} )} ,$$

Where, Phase₁ is a first measured impedance phase value at a first frequency value (Frequency₁) of 100 kHz and Phase₂ is a second measured impedance phase value at a second frequency value (Frequency₂) of 500 kHz.

In an exemplary implementation, the method may further include generating a calibration dataset. In an exemplary implementation, generating the calibration dataset may include generating the reference impedance value and generating the reference IPS.

In an exemplary implementation, generating the reference impedance value may include measuring a set of Z_{1 kHz} values from a plurality of masses associated with a respective plurality of persons and determining the reference impedance value equal to a maximum value of the measured set of Z_{1 kHz} values.

In an exemplary implementation, generating the reference IPS may include calculating, utilizing one or more processors, a set of IPS values from the plurality of masses, determining cancerous status of each mass of the plurality of masses by applying a pathological assay to each mass, assigning the determined status of each mass to the respective calculated IPS value, classifying the set of IPS values into three IPS ranges based on the determined cancerous status of each mass of the plurality of masses, and determining the reference IPS based on the classified set of IPS values.

In an exemplary embodiment, the determined cancerous status may include one of a benign state, a cancerous state, and a precancerous state, based on result of the applied pathological assay. In an exemplary implementation, classifying the set of IPS values into three IPS ranges may include classifying the set of IPS values into one of a benign IPS range, a precancerous IPS range, and a cancerous IPS range. In an exemplary embodiment, the benign IPS range may include a first range of IPS values assigned as being of the benign state. In an exemplary embodiment, the precancerous IPS range may include a second range of IPS values assigned as being of the precancerous state. In an exemplary embodiment, the cancerous IPS range may include a third range of IPS values assigned as being of the cancerous state. In an exemplary implementation, determining the reference IPS based on the classified set of IPS values may include determining the reference IPS equal to a value between a minimum IPS value among IPS values of the benign IPS range and a maximum IPS value among IPS values of both the precancerous IPS range and the cancerous IPS range.

In an exemplary implementation, the suspected mass may include a suspected mass to be cancerous or precancerous that may be located in a breast tissue. In an exemplary embodiment, the reference impedance value may include an impedance value of 1500Ω and the reference IPS may include an IPS value equal to zero. In an exemplary implementation, detecting the cancerous status of the suspected mass may include detecting the suspected mass being a cancerous (malignant) breast lesion if the calculated IPS is less than an IPS value of −2, or detecting the suspected mass is a precancerous (borderline or high-risk) breast lesion if the calculated IPS is in a range of IPS value between −2 and 0.

In an exemplary implementation, the suspected mass may include a suspected nodule to be cancerous or precancerous that may be located in a thyroid gland. In an exemplary implementation, the reference impedance value may include an impedance value of 2000Ω, and the reference IPS may include an IPS value of 2. In an exemplary implementation, detecting the cancerous status of the suspected mass may include detecting the suspected nodule is one of a cancerous thyroid nodule and a precancerous thyroid nodule if the calculated IPS is less than an IPS value of 2.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A shows a schematic view of an exemplary electrical probe, consistent with one or more exemplary embodiments of the present disclosure.

FIG. 1B illustrates a schematic exploded view of an exemplary electrical probe, consistent with one or more exemplary embodiments of the present disclosure.

FIG. 1C illustrates another schematic exploded view of an exemplary electrical probe, consistent with one or more exemplary embodiments of the present disclosure.

FIG. 2 shows an exemplary system for real-time, in-vivo, and non-invasive status detection of an exemplary suspected mass to be cancerous/precancerous in a living body, consistent with one or more exemplary embodiments of the present disclosure.

FIG. 3A shows an exemplary method for real-time, in-vivo, and non-invasive detecting cancerous status of an exemplary suspected mass to be cancerous/precancerous in a living body, consistent with one or more exemplary embodiments of the present disclosure.

FIG. 3B shows an exemplary implementation of an exemplary method for real-time, in-vivo, and non-invasive detecting cancerous status of an exemplary suspected mass to be cancerous/precancerous in a living body, consistent with one or more exemplary embodiments of the present disclosure.

FIG. 4A shows an exemplary implementation of an exemplary method for preparing an electrical probe, consistent with one or more exemplary embodiments of the present disclosure.

FIG. 4B shows an exemplary implementation of an exemplary method for recording an electrical impedance spectroscopy (EIS) from an exemplary suspected mass, consistent with one or more exemplary embodiments of the present disclosure.

FIG. 5 illustrates an example computer system in which an embodiment of the present disclosure, or portions thereof, may be implemented as computer-readable code, consistent with one or more exemplary embodiments of the present disclosure.

FIG. 6A shows comparison of frequency responses of muscular (normal) and cancerous tissues of an exemplary mouse model, consistent with one or more exemplary embodiments of the present disclosure.

FIG. 6B shows comparative histopathological Hematoxylin and Eosin (H&E) assays of an exemplary healthy tissue and an exemplary cancerous tissue of an exemplary mouse model, consistent with one or more exemplary embodiments of the present disclosure.

FIG. 7A shows impedance magnitude and phase diagram for benign, high-risk benign (borderline), and malignant breast lesions, consistent with one or more exemplary embodiments of the present disclosure.

FIG. 7B shows an exemplary scale bar for breast lesion classification with IPS and Ultra Sonography (US) images and H&E assays for a low-risk benign lesion BI-RADS4a, a borderline breast disease (complex sclerosing adenosis) BI-RADS4a, and a malignant lesion with BI-RADS5, consistent with one or more exemplary embodiments of the present disclosure.

FIG. 8 shows histological patterns of H&E assay for patient ID87 with BI-RADS: B4a and IPS=−2.2 (malignant), core needle biopsy (CNB) result: papillary lesion with ADH, Permanent pathology: DCIS, 100×; patient ID 95 with BI-RADS: B4a, IPS=−1.7 (borderline), CNB result: FEL in favor of FA, Permanent pathology: FA, moderate typical ductal hyperplasia, extensive area of CCC and CCH, 400×; patient ID49 with BI-RADS: B4a, IPS=−0.7 (borderline), CNB result: proliferative FCC, Permanent pathology: Sclerosing papillary lesion with lactational changes, complex SA, FCC, UDH, and simple adenosis, 100×; and patient ID63 with BI-RADS: B4a, IPS=−2.7, CNB result: FEL in favor of benign phyllodes tumor, Permanent pathology: Cellular FA, 100×, consistent with one or more exemplary embodiments of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings. The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Herein, an exemplary probe, system and method for detecting cancerous status of an exemplary lesion or part of a tissue is described based on an Electrical Impedance Spectroscopy (EIS) recorded from an exemplary lesion or part of a tissue. An exemplary recorded EIS, utilizing an exemplary probe and system, may include a frequency-dependent response of a biological tissue to an electrical stimulation. Electrical impedance properties of malignant tissues may be different from benign or normal ones. Such differences may be initiated from non-similar cellular water and electrolyte content, changes in cell membrane permeability (due to changed phospholipids to lipoproteins ratio in the membrane), charges, orientation, and packing density of cells of a malignant tissue and a benign one. In other words, complex electrical impedance of biological tissues in response to an alternating electrical stimulation may be related to composition, structure, health status, and physiological or pathological properties of biological tissues. Exemplary probe, system and method describe herein may be utilized to distinct excision-requiring (cancerous or precancerous) tumors or masses from low-risk benign ones. In an exemplary embodiment, excision-requiring tumors or masses may refer to cancerous or precancerous tumors or masses. In an exemplary embodiment, a cancerous mass may refer to a malignant mass or tumor. In an exemplary embodiment, a precancerous mass may refer to one of a mass or tumor in an early stage of cancer progression, a borderline mass or tumor, and a high-risk mass or tumor. In an exemplary embodiment, a high-risk mass or tumor may refer to a mass that has a high possibility to transform to a precancerous or cancerous mass in future. In an exemplary embodiment, a high-risk mass mostly occur simultaneously with a cancer, for example, in a cancer patient's body but not necessarily in a known cancer-involved tissue or organ of the cancer patient.

Borderline Breast Diseases (BBD) are some breast lesions that may result in diagnostic uncertainty when detected in needle biopsies. This uncertainty may arise from difficulties in differentiating borderline breast lesions from malignant tumors or coexistence probability of borderline breast lesions in malignant ones. Although advances in breast imaging technologies have positively impacted diagnosis of high-risk proliferative breast disease in an early stage, borderline breast lesions are still challenging in pathological detections, even in tissue sections taken from surgically excised lesions. It is more critical in pathological evaluations of cellular samples or small fragmented tissues obtained from Fine Needle Aspiration (FNA) or Core Needle Biopsies (CNB). Herein, an exemplary probe, system and method is disclosed for finding BI-RADS4 tumors among missed scored BI-RADS3 lesions. An exemplary impedance-based calibration database may be generated to distinguish breast lesions with borderline or high-risk nature that may be required for excision based on the pathological guidelines from low-risk benign lesions.

Herein, an exemplary probe, system, and method is disclosed for in-vivo EIS recording of a living tissue or lesion or a suspected mass within a living body to be excision-requiring due to the mass being a cancerous, or precancerous mass. An exemplary probe may include two needle electrodes, where one exemplary electrode may be placed inside another exemplary electrode; allowing for more precise and less invasive live impedance measurements from a living tissue. An exemplary inner electrode may be movable inside an exemplary outer electrode; allowing for tunable in-situ inner electrode size depending on width and location of an exemplary tumor or lesion. An exemplary probe may include an integrated needle including two medical grade stainless steel electrodes inserted into exemplary lesions, such as breast lesions.

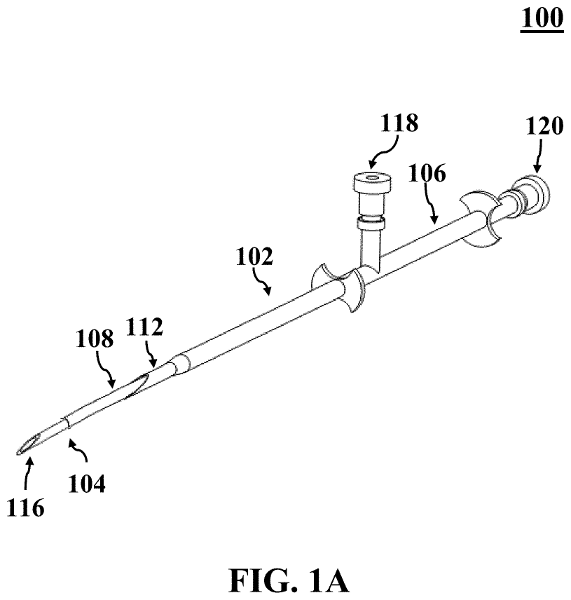

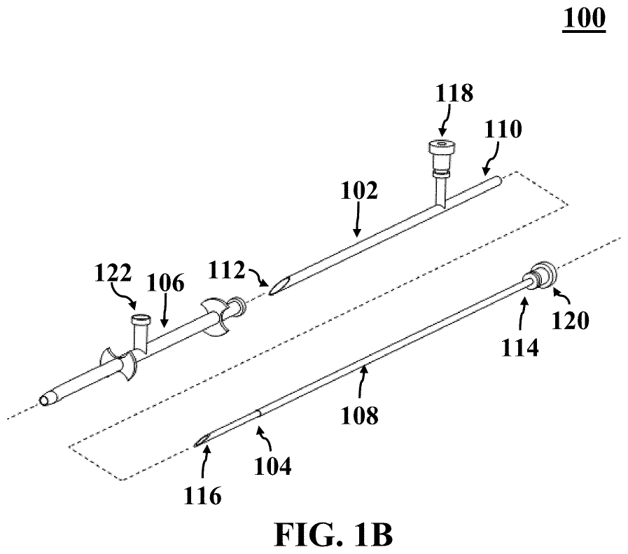

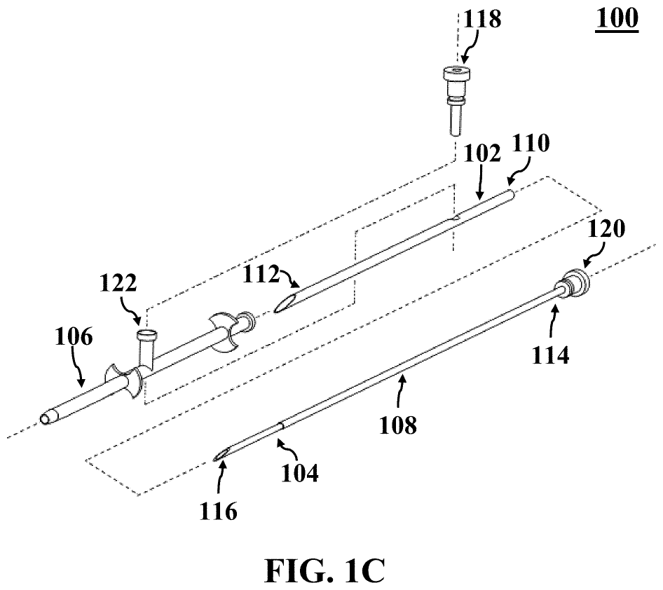

FIG. 1A shows a schematic view of exemplary electrical probe 100, consistent with one or more exemplary embodiments of the present disclosure. Furthermore, FIG. 1B illustrates a schematic exploded view of exemplary electrical probe 100, consistent with one or more exemplary embodiments of the present disclosure. Additionally, FIG. 1C illustrates another schematic exploded view of exemplary electrical probe 100, consistent with one or more exemplary embodiments of the present disclosure. In an exemplary embodiment, electrical probe 100 may include first electrode 102 and second electrode 104. In an exemplary embodiment, second electrode 104 may be placed inside first electrode 102.

In an exemplary embodiment, first electrode 102 and second electrode 104 may include a couple of electrical electrodes configured to be utilized to conduct electrical measurements, such as electrical impedance measurement of a sample. In an exemplary embodiment, first electrode 102 may include a first electrically conductive needle. In an exemplary embodiment, first electrode 102 may include a needle made of an electrically conductive biocompatible material, for example, stainless steel. In an exemplary embodiment, first electrode 102 may include a hollow needle; allowing for placing second electrode 104 inside the respective hollow part. In an exemplary embodiment, first electrode 102 may include a needle of a peripheral venous catheter or an intravenous (IV) cannula. In an exemplary embodiment, first electrode 102 may include a needle of a peripheral venous catheter or an IV cannula with a gauge size of 14 or more, for example, at least one of gauge 16, gauge 18, and gauge 20 and so on. In an exemplary embodiment, first electrode 102 may include a needle of a peripheral venous catheter or an IV cannula with an external diameter of about 2.1 mm or less. In an exemplary embodiment, first electrode 102 may include a medical-grade needle of a peripheral venous catheter or an IV cannula with a size of gauge 14 that may have an external diameter of about 2.1 mm and a length of about 55 mm.

In an exemplary embodiment, second electrode 104 may comprise a second electrically conductive needle including a medical-grade needle. In an exemplary embodiment, the second electrically conductive needle may have an external diameter less than an internal diameter of the first electrically conductive needle; allowing for moving the second electrically conductive needle inside the first electrically conductive needle. In an exemplary embodiment, the second electrically conductive needle may comprise a hollow needle. In an exemplary embodiment, second electrode 104 may be placed inside first electrode 102. In an exemplary embodiment, second electrode 104 may be movable in a longitudinal direction of length of first electrode 102 along length of first electrode 102 while being inside first electrode 102.

In an exemplary embodiment, second electrode 104 may include a needle made of an electrically conductive biocompatible material, for example, medical-grade stainless steel. In an exemplary embodiment, second electrode 104 may comprise a medical-grade stainless steel needle with a gauge size of 20 or more. In an exemplary embodiment, second electrode 104 may comprise a spinal cannula needle with a gauge size of 20. In an exemplary embodiment, the second electrically conductive needle may have a thickness between about 300 μm and about 700 μm.

In an exemplary embodiment, first electrode 102 may comprise first distal end portion 112 and first proximal end portion 110. In an exemplary embodiment, second electrode 104 may comprise second distal end portion 116 and second proximal end portion 114. In an exemplary embodiment, first distal end portion 112 and second distal end portion 116 may define (comprise) a respective length of first electrode 102 and second electrode 104 to be inserted into a sample or to be put in contact with the sample. In an exemplary embodiment, first distal end portion 112 and second distal end portion 116 may be configured to be put in contact with a biological sample. In an exemplary embodiment, the biological sample may include a suspected mass in a living body/tissue. In an exemplary embodiment, the “suspected mass” may refer to a mass suspected to be cancerous or precancerous so then the suspected mass may be excision-requiring if the suspected mass is detected as a precancerous mass or a cancerous mass. In an exemplary embodiment, a suspected mass may refer to a mass in the living tissue that is suspected to be either cancerous or precancerous, that is therefore required to be excised. In an exemplary embodiment, first distal end portion 112 and second distal end portion 116 may be configured to be inserted into the suspected mass. In an exemplary embodiment, both of first electrode 102 and second electrode 104 may have sharp pointed tips at their receptive distal ends 112 and 116; leading to simple and less-invasive insertion of first electrode 102 and second electrode 104 into a sample.

In an exemplary embodiment, second distal end portion 116 may be placed inside or outside of first electrode 102. In an exemplary embodiment, second distal end portion 116 may be placed (adjusted to be placed) outside of first electrode 102 while putting first distal end portion 112 and second distal end portion 116 in contact with the biological sample. In an exemplary embodiment, a length of second distal end portion 116 may be tunable by moving second electrode 104 in longitudinal direction inside first electrode 102 depending on a desired depth of a sample to be put in contact with second electrode 104. In an exemplary embodiment, the length of second distal end portion 116 may be tunable by pushing and/or pulling second electrode 104 in longitudinal direction inside first electrode 102 depending on the desired depth of the sample to be put in contact with second electrode 104. In an exemplary embodiment, a length of second distal end portion 116 that may be placed outside of first electrode 102 may be adjusted based on a depth of a target location of a suspected mass to be put in contact with first electrode 102 and second electrode 104. In an exemplary embodiment, the length of second distal end portion 116 that may be placed outside of first electrode 102 may be adjusted by pushing and/or pulling second electrode 104 in longitudinal direction inside first electrode 102.

In an exemplary embodiment, electrical probe 100 may further include two respective electrically insulating layers 106 and 108 coated on respective outer surfaces of first electrode 102 and second electrode 104. In an exemplary embodiment, electrically insulating layers 106 and 108 may be partially coated on respective outer surfaces of first electrode 102 and second electrode 104 so that first distal end portion 112 and second distal end portion 116 may remain uncoated. In an exemplary embodiment, electrically insulating layers 106 and 108 may prevent transferring electricity to non-targeted internal organs and skin throughout a length of an insertion path of first electrode 102 and second electrode 104 within a patient's body; thereby, result in preventing transmission of electricity to the non-targeted internal organs and skin. Furthermore, electrically insulating layers 106 and 108 may prevent direct contact of first electrode 102 and second electrode 104 with each other; thereby, resulting in preventing electrical short circuits during an electrical measurement process, for example, in an exemplary electrical impedance measurement. In an exemplary embodiment, electrically insulating layers 106 and 108 may comprise first electrically insulating layer 106 coated around first electrode 102 except first distal end portion 112 and second electrically insulating layer 108 coated around second electrode 104 except second distal end portion 116; allowing for insulating portions of a sample or a patient's body adjacent to the suspected mass from electrical signals that may be applied to electrical probe 100.

In an exemplary embodiment, first electrically insulating layer 106 may comprise a tubular cover placed around first electrode 102 except first distal end portion 112. In an exemplary embodiment, first electrically insulating layer 106 may comprise a plastic cannula covered first electrode 102 except first distal end portion 112. In an exemplary embodiment, first electrically insulating layer 106 may comprise a layer of an electrically insulating material coated around first electrode 102 except the distal end portion 112; allowing for putting first distal end portion 112 of first electrode 102 in contact with a sample. The first electrically insulating layer 106 may be configured to protect surrounding tissue(s) of an exemplary suspected mass from being in contact with first electrode 102 to prevent possible side effects of an electrical signal applied to and/or received from electrical probe 100, that is, due to the fact that first electrically insulating layer 106 may comprise an electrically insulating material, surround tissue of an exemplary suspected mass may be insulated from first electrode 102. In an exemplary embodiment, a left ventricular (LV) cannula needle or an IV cannula needle with gauge 14 may be utilized as first electrode 102 with electrically insulating layer 106.

In an exemplary embodiment, second electrically insulating layer 108 may comprise a layer of an electrically insulating material that may be placed between first electrode 102 and second electrode 104. In an exemplary embodiment, second electrically insulating layer 108 may comprise a layer of an electrically insulating material coated around second electrode 104 except second distal end portion 116. The second distal end portion 116 may be kept uncoated as an electrically interactive length of second electrode 104 that may be configured to be put in contact with an exemplary suspected mass. The second electrically insulating layer 108 may be configured to be electrically insulating first electrode 102 and second electrode 104 from each other in order to prevent possible electrical short circuits while applying an electrical signal to electrical probe 100 and/or receiving an electrical signal from electrical probe 100. Furthermore, second electrically insulating layer 108 may be configured to be electrically insulating electrically insulating surrounding tissue(s) of an exemplary suspected mass from being in contact with second electrode 104 in order to prevent possible side effects of an electrical signal applied to and/or received from electrical probe 100.

Referring to FIGS. 1A-1C, electrical probe 100 may further comprise two electrical connectors 118 and 120. In an exemplary embodiment, first electrical connector 118 may be attached onto a surface of first electrode 102 adjacent to first proximal end portion 110 of first electrode 102. In an exemplary embodiment, first electrically insulating layer 106 may comprise exemplary opening 122 adjacent to first proximal end portion 110. In an exemplary embodiment, first electrical connector 118 may be passed through opening 122 and attached onto the surface of first electrode 102. In an exemplary embodiment, first electrical connector 118 may be configured to connect first electrode 102 to an exemplary electrical device via connecting first electrical connector 118 to the electrical device. In an exemplary embodiment, first electrical connector 118 may be configured to be connected to an impedance analyzer device.

In an exemplary embodiment, second electrical connector 120 may be attached onto a surface of second electrode 104 adjacent to second proximal end portion 114 of second electrode 104. In an exemplary embodiment, second electrical connector 120 may be configured to connect second electrode 104 to an exemplary electrical device via connecting second electrical connector 120 to the electrical device. In an exemplary embodiment, second electrical connector 120 may be configured to be connected to an impedance analyzer device.

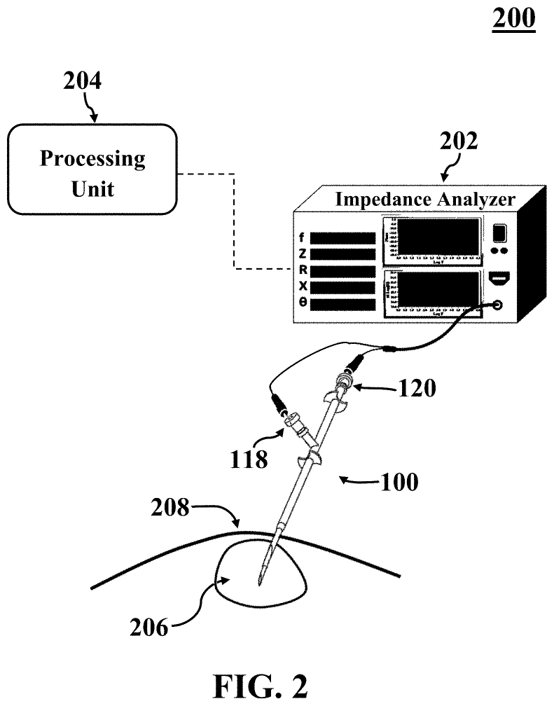

In an exemplary implementation of the present disclosure, exemplary electrical probe 100 may be utilized via an exemplary method and an exemplary system for real-time, in-vivo, and non-invasive status detection of a suspected mass to be an excision-requiring cancerous or precancerous mass in a living body. In an exemplary embodiment, electrical probe 100 may be utilized via an exemplary method in an exemplary system for determining status of a suspected mass to be an excision-requiring cancerous or precancerous mass via measuring, monitoring and analyzing electrical impedance and electrical impedance phase of the suspected mass. FIG. 2 shows exemplary system 200 for real-time, in-vivo, and non-invasive status detection of exemplary suspected mass 206 to be cancerous/precancerous in a living body, consistent with one or more exemplary embodiments of the present disclosure. In an exemplary embodiment, exemplary system 200 may comprise electrical probe 100, impedance analyzer device 202, and processing unit 204. In an exemplary embodiment, impedance analyzer device 202 may be configured to be electrically connected to electrical probe 100 and processing unit 204.

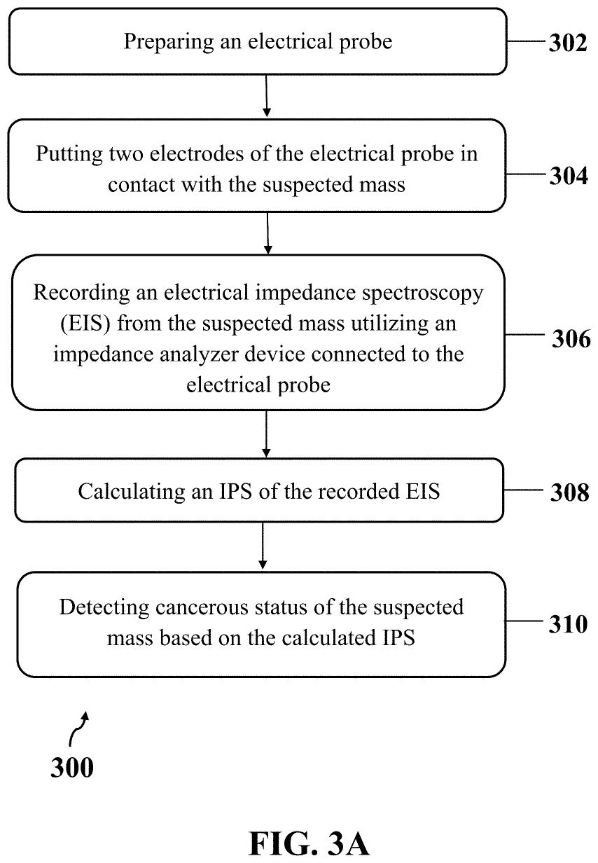

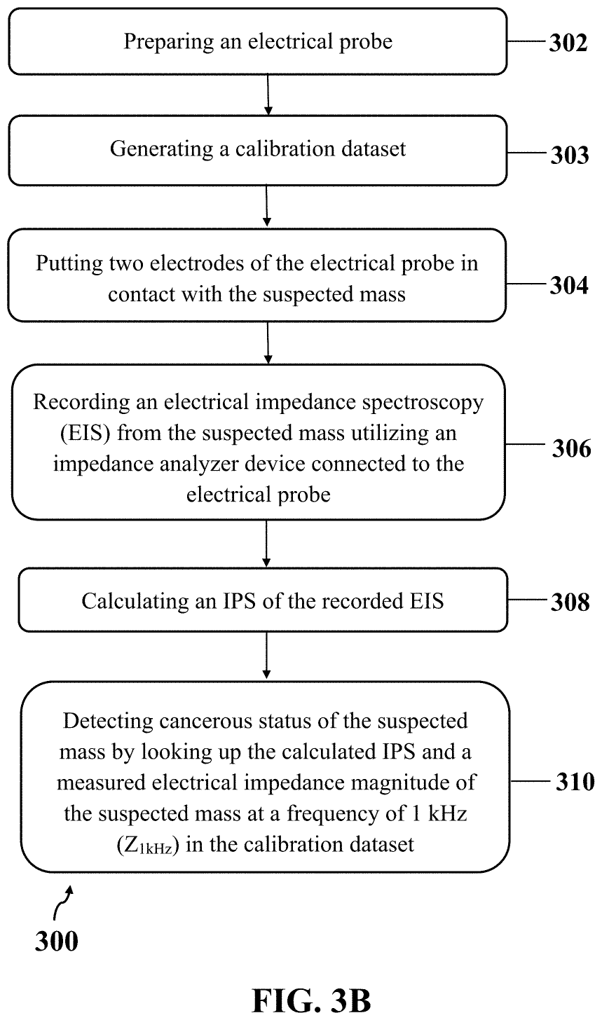

In an exemplary implementation, exemplary electrical probe 100 and exemplary system 200 may be utilized via an exemplary method for real-time in-vivo status detection of suspected mass 206 to be an excision-required cancerous or precancerous mass in a living body. FIG. 3A shows exemplary method 300 for real-time, in-vivo, and non-invasive detecting cancerous status of exemplary suspected mass 206 to be cancerous/precancerous in a living body, consistent with one or more exemplary embodiments of the present disclosure. In an exemplary embodiment, exemplary steps of method 300 are explained in context of elements of FIGS. 1 and 2 as exemplary explanations but may be utilized for similar masses, etc. In an exemplary implementation, method 300 may comprise preparing electrical probe 100 (step 302), putting two electrodes of electrical probe 100 in contact with suspected mass 206 (step 304), recording an electrical impedance spectroscopy (EIS) from suspected mass 206 utilizing impedance analyzer device 202 (step 306), calculating an impedance phase slope (IPS) of the recorded EIS from suspected mass 206 (step 308), and detecting cancerous status of suspected mass 206 based on the calculated IPS (step 310).

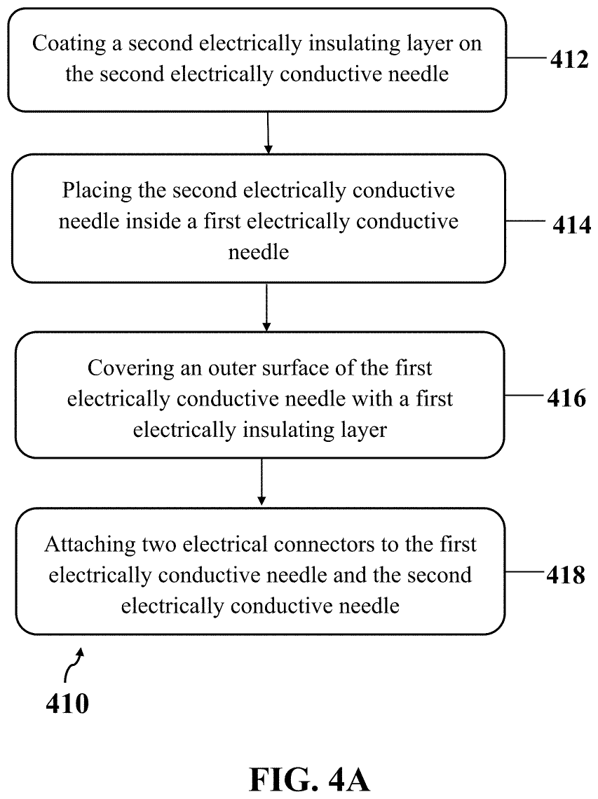

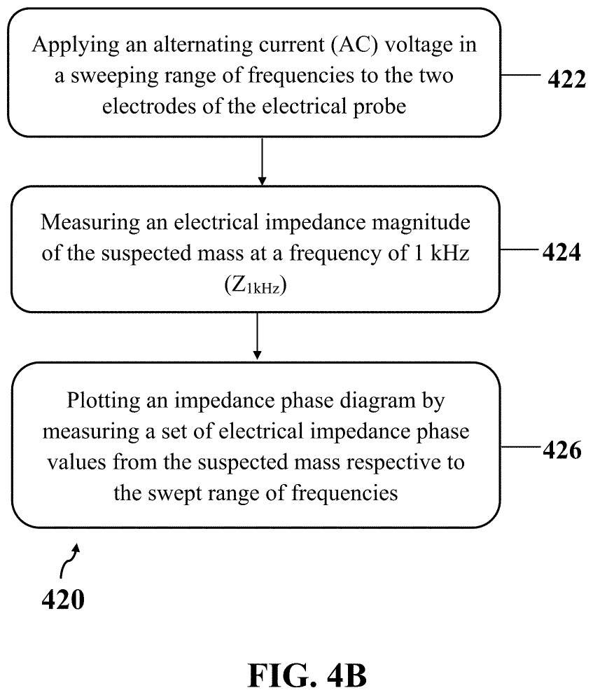

In detail, step 302 may comprise preparing electrical probe 100. FIG. 4A shows an exemplary implementation of exemplary method 410 for preparing electrical probe 100 (step 302), consistent with one or more exemplary embodiments of the present disclosure. In an exemplary implementation, method 410 for preparing electrical probe 100 may comprise forming second electrode 104 by coating second electrically insulating layer 108 on the second electrically conductive needle except second distal end portion 116 of the second electrically conductive needle (step 412), placing the second electrically conductive needle inside the first electrically conductive needle (step 414), covering an outer surface of the first electrically conductive needle with first electrically insulating layer 106 except first distal end portion 112 of the first electrically conductive needle (step 416), and attaching two electrical connectors 118 and 120 to the first electrically conductive needle and the second electrically conductive needle (step 418).

In an exemplary implementation, step 412 may comprise coating second electrically insulating layer 108 on the second electrically conductive needle except second distal end portion 116 of the second electrically conductive needle. In an exemplary implementation, coating second electrically insulating layer 108 on the second electrically conductive needle may comprise adhering a thin layer of biocompatible plastic cannula with a thickness between about 200 μm and 500 μm on the second electrically conductive needle except second distal end portion 116 of the second electrically conductive needle. In an exemplary implementation, coating second electrically insulating layer 108 on the second electrically conductive needle may comprise adhering the thin layer of biocompatible plastic cannula on the second electrically conductive needle and peeling a length of the adhered biocompatible plastic cannula on second distal end portion 116 of the second electrically conductive needle. In an exemplary implementation, adhering the thin layer of biocompatible plastic cannula on the second electrically conductive needle may be done by heating the thin layer of biocompatible plastic cannula and/or the second electrically conductive needle. In an exemplary implementation, adhering the thin layer of biocompatible plastic cannula on the second electrically conductive needle may comprise adhering a hot thin layer of biocompatible plastic cannula on the second electrically conductive needle.

In an exemplary implementation, attaching two electrical connectors 118 and 120 to the first electrically conductive needle and the second electrically conductive needle (step 418) may comprise attaching first electrical connector 118 onto a surface of the first electrically conductive needle adjacent to first proximal end portion 110 of the first electrically conductive needle and attaching second electrical connector 120 onto second proximal end portion 114 of the second electrically conductive needle.

Referring again to FIG. 3A, step 304 may comprise putting two electrodes 102 and 104 of electrical probe 100 in contact with suspected mass 206 with reference to FIG. 2. In an exemplary implementation, putting two electrodes 102 and 104 of electrical probe 100 in contact with suspected mass 206 may comprise inserting first electrode 102 and second electrode 104 of electrical probe 100 into suspected mass 206. In an exemplary implementation, putting two electrodes 102 and 104 of electrical probe 100 in contact with suspected mass 206 may comprise non-invasive putting two electrodes 102 and 104 of electrical probe 100 in contact with suspected mass 206 by inserting first electrode 102 through an exemplary portion of skin 208 into suspected mass 206 and pushing second electrode 104 through first electrode 102 into suspected mass 206. In an exemplary embodiment, exemplary portion of skin 208 may be located adjacent to suspected mass 206. Hence, a non-invasive contact between exemplary electrodes 102 and 104 and exemplary suspected mass 206 may be achieved via insertion of electrode 102 through skin 208 into suspected mass 206.

In an exemplary implementation, putting two electrodes 102 and 104 of electrical probe 100 in contact with suspected mass 206 may comprise putting first distal end portion 112 of first electrode 102 and second distal end portion 116 of second electrode 104 of electrical probe 100 in contact with suspected mass 206. In an exemplary implementation, putting two electrodes 102 and 104 of electrical probe 100 in contact with suspected mass 206 may comprise inserting first distal end portion 112 of first electrode 102 through an exemplary portion of skin 208 inside suspected mass 206 and pushing second distal end portion 116 of second electrode 104 through first electrode 102 inside suspected mass 206. In an exemplary implementation, putting two electrodes of electrical probe 100 in contact with suspected mass 206 may be carried out while conducting sonography imaging from a location of suspected mass 206; allowing for precise insertion of first electrode 102 and second electrode 104 into suspected mass 206.

In an exemplary implementation, putting two electrodes 102 and 104 of electrical probe 100 in contact with suspected mass 206 may comprise inserting first distal end portion 112 of first electrode 102 and second distal end portion 116 of second electrode 104 into suspected mass 206 through an exemplary portion of skin 208 even though suspected mass 206 located at deep locations respective to exemplary portion of skin 208. In an exemplary implementation, putting two electrodes 102 and 104 of electrical probe 100 in contact with suspected mass 206 may comprise inserting first distal end portion 112 of first electrode 102 and second distal end portion 116 of second electrode 104 into suspected mass 206 located at a distance from exemplary portion of skin 208 up to about 10 cm.

Moreover, referring to FIG. 3A, step 306 may comprise recording an electrical impedance spectroscopy (EIS) from an exemplary mass utilizing an exemplary impedance analyzer device. In an exemplary embodiment, step 306 may comprise recording an electrical impedance spectroscopy (EIS) from exemplary suspected mass 206 utilizing exemplary impedance analyzer device 202. FIG. 4B shows an exemplary implementation of exemplary method 420 for recording an electrical impedance spectroscopy (EIS) from exemplary suspected mass 206 (step 306), consistent with one or more exemplary embodiments of the present disclosure. In an exemplary implementation, method 420 for recording an electrical impedance spectroscopy (EIS) from exemplary suspected mass 206 may comprise applying an alternating current (AC) voltage in a sweeping range of frequencies to exemplary two electrodes 102 and 104 of electrical probe 100 (step 422), measuring an electrical impedance magnitude of suspected mass 206 at a frequency magnitude of 1 kHz (Z_{1 kHz}) (step 424), and plotting an impedance phase diagram by measuring a set of electrical impedance phase values from suspected mass 206 respective to the swept range of frequencies (step 426).

In an exemplary implementation, step 422 may comprise applying an AC voltage in a sweeping range of frequencies to exemplary two electrodes 102 and 104 of electrical probe 100 utilizing impedance analyzer device 202 where two electrodes 102 and 104 may be placed inside suspected mass 206. In an exemplary implementation, applying an AC voltage in a sweeping range of frequencies to exemplary two electrodes 102 and 104 in contact with suspected mass 206 may comprise connecting first proximal end portion 110 of first electrode 102 and second proximal end portion 114 of second electrode 104 of electrical probe 100 to impedance analyzer device 202 and applying an AC voltage in a sweeping range of frequencies between about 1 Hz and about 1 MHz to two electrodes 102 and 104 utilizing impedance analyzer device 202. In an exemplary implementation, connecting first proximal end portion 110 of first electrode 102 and second proximal end portion 114 of second electrode 104 of electrical probe 100 to impedance analyzer device 202 may comprise connecting first proximal end portion 110 and second proximal end portion 114 to impedance analyzer device 202 via first electrical connector 118 and second electrical connector 118, respectively. In an exemplary implementation, applying an AC voltage in a sweeping range of frequencies between about 1 Hz and about 1 MHz to two electrodes 102 and 104 may comprise applying an AC voltage in a sweeping range of frequencies between about 1 kHz and about 500 kHz to two electrodes 102 and 104.

In an exemplary implementation, step 424 may comprise measuring an electrical impedance magnitude of suspected mass 206 at a frequency magnitude of 1 kHz (Z_{1 kHz}). In an exemplary implementation, measuring Z_{1 kHz} of suspected mass 206 may comprise recording a magnitude of a real part of electrical impedance of suspected mass 206 at a frequency magnitude of 1 kHz while applying an AC voltage to two electrodes 102 and 104 being in contact with suspected mass 206.

In an exemplary implementation, Z_{1 kHz} of suspected mass 206 may be utilized as a determinative parameter for detecting a cancerous status of suspected mass 206. In an exemplary embodiment, suspected mass 206 may be detected as a cancerous solid tumor or a precancerous mass if the measured Z_{1 kHz} is less than a reference impedance value. In another exemplary embodiment, suspected mass 206 may be detected as at least one of a normal tissue, a benign lesion, a fat necrotic region, and a benign glandular region if the measured Z_{1 kHz} is more than the reference impedance value. In an exemplary embodiment, exemplary method 300 may be calibrated for each organ or specific part of a living body regarding the reference impedance value for classifying an exemplary suspected mass 206 located within a respective organ or specific part of a living body based on the measured Z_{1 kHz}. In an exemplary embodiment, the reference impedance value may comprise a distinctive value for each organ or specific part of a living body, such as breast, thyroid, etc.

In an exemplary implementation, step 426 may comprise plotting an impedance phase diagram by measuring a set of electrical impedance phase values from suspected mass 206 respective to the swept range of frequencies. In an exemplary implementation, plotting an impedance phase diagram may comprise plotting the set of the measured electrical impedance phase values from suspected mass 206 versus a respective set of the swept range of frequencies while applying an AC voltage to two electrodes 102 and 104 being in contact with suspected mass 206 in the swept range of frequencies.

In an exemplary implementation, steps 422 to 426 of exemplary method 420 for recording an electrical impedance spectroscopy (EIS) from exemplary suspected mass 206 (step 306) may be done during a time period in a range between about 3 second and about 30 seconds. In an exemplary implementation, steps 422 to 426 of exemplary method 420 for recording an electrical impedance spectroscopy (EIS) from exemplary suspected mass 206 (step 306) may be done in less than 10 seconds.

Moreover, referring to FIG. 3A, step 308 may comprise calculating an impedance phase slope (IPS) of the recorded EIS from suspected mass 206. In an exemplary implementation, calculating an IPS of the recorded EIS from suspected mass 206 may comprise calculating an IPS of the plotted impedance phase diagram in a frequency range between about 100 kHz and about 500 kHz if the measured Z_{1 kHz} is less than the reference impedance value. In an exemplary implementation, calculating the IPS may comprise:

$$IPS = \frac{{Phase}_{2} - {Phase}_{1}}{log ( {Frequency}_{2} ) - log ( {Frequency}_{1} )}$$

Where, Phase₁ may be a first measured impedance phase value at a first frequency value (Frequency₁) of 100 kHz, and Phase₂ may be a second measured impedance phase value at a second frequency value (Frequency₂) of 500 kHz.

In an exemplary implementation, calculated IPS of suspected mass 206 may be utilized as an exemplary determinative parameter for detecting cancerous status of suspected mass 206. In an exemplary embodiment, there may be a different ranges of slope of electrical impedance phase diagram (IPS) calculated in a frequency range between about 100 kHz and about 500 kHz for benign and/or non-cancerous masses in comparison with cancerous or precancerous masses due to a difference in electrical properties of benign and/or non-cancerous masses in comparison with cancerous or precancerous masses. Accordingly, the calculated IPS may be the most important parameter in classifying tissues utilizing electrical probe 100 and system 200 by conducting exemplary method 300. In an exemplary embodiment, normal (healthy) tissues or benign (non-cancerous) lesions may show an exemplary calculated IPS value more than a reference IPS value, whereas a cancerous or precancerous mass may show an exemplary calculated IPS value less than the reference IPS value that may be an effective classification parameter. In an exemplary embodiment, a value of the reference IPS value may depend on a type or location of a tissue, organ or part of a living body where suspected mass 206 may be located therein. In an exemplary embodiment, the reference IPS value may comprise a distinctive value for each organ or specific part of a living body, such as breast, thyroid, etc.

Furthermore, referring to FIG. 3A, step 310 may comprise detecting a cancerous status of suspected mass 206 based on the calculated IPS. In an exemplary implementation, detecting the cancerous status of suspected mass 206 based on the calculated IPS may comprise detecting suspected mass 206 being a cancerous mass or a precancerous mass, or detecting suspected mass 206 being a benign (healthy) region. In an exemplary implementation, detecting the cancerous status of suspected mass 206 based on the calculated IPS may comprise detecting suspected mass 206 being an excision-requiring cancerous or precancerous mass if the calculated IPS is less than a reference IPS. In an exemplary implementation, detecting suspected mass 206 being an excision-requiring mass may comprise detecting suspected mass 206 is at least one of a cancerous mass and a precancerous mass. In an exemplary implementation, detecting the cancerous status of suspected mass 206 based on the calculated IPS may comprise detecting suspected mass 206 being a benign region responsive to the calculated IPS being more than the reference IPS.

FIG. 3B shows another exemplary implementation of exemplary method 300 for real-time, in-vivo, and non-invasive detecting cancerous status of an exemplary suspected mass to be cancerous/precancerous in a living body, consistent with one or more exemplary embodiments of the present disclosure. Referring to FIG. 3B, exemplary method 300 may further comprise step 303 comprising generating a calibration dataset. In an exemplary implementation, generating the calibration dataset (step 303) may comprise generating the reference impedance value and generating the reference IPS.

In an exemplary implementation, generating the reference impedance value may comprise measuring a set of Z_{1 kHz} values from a plurality of masses associated with a respective plurality of persons and determining the reference impedance value equal to a maximum value of the set of measured Z_{1 kHz} values utilizing one or more processors. In an exemplary implementation, measuring the set of Z_{1 kHz} values for each mass of the plurality of masses may be done via a process comprising steps 304, 422 and 424 described hereinabove.

In an exemplary implementation, generating the reference IPS may comprise calculating, utilizing one or more processors, a set of IPS values from the plurality of masses, determining cancerous status of each mass of the plurality of masses by applying a pathological assay to each mass, assigning the determined status of each mass to the respective calculated IPS value utilizing one or more processors, classifying the set of IPS values into three IPS ranges based on the determined cancerous status of each mass of the plurality of masses utilizing one or more processors, and determining the reference IPS based on the classified set of IPS values utilizing one or more processors.

In an exemplary implementation, the determined cancerous status may comprise one of a benign state, a cancerous state, and a precancerous state based on result of the applied pathological assay. In an exemplary implementation, classifying the set of IPS values into three IPS ranges may comprise classifying the set of IPS values into one of a benign IPS range, a precancerous IPS range, and a cancerous IPS range. In an exemplary embodiment, the benign IPS range may include a first range of IPS values assigned as being of the benign state. In an exemplary embodiment, the precancerous IPS range may include a second range of IPS values assigned as being of the precancerous state. In an exemplary embodiment, the cancerous IPS range may include a third range of IPS values assigned as being of the cancerous state. In an exemplary implementation, determining the reference IPS based on the classified set of IPS values may comprise determining the reference IPS equal to a value between a minimum IPS value among IPS values of the benign IPS range and a maximum IPS value among IPS values of both the precancerous IPS range and the cancerous IPS range. In an exemplary implementation, determining the reference IPS based on the classified set of IPS values may comprise determining the reference IPS equal to a minimum IPS value among IPS values of the benign IPS range. In an exemplary implementation, determining the reference IPS based on the classified set of IPS values may comprise determining the reference IPS equal to a maximum IPS value among IPS values of both the precancerous IPS range and the cancerous IPS range.

In an exemplary embodiment, classifying the set of IPS values into three IPS ranges may further comprise integrating the precancerous IPS range and the cancerous IPS range together, and forming a cancerous/precancerous IPS range in a case of which the second range of IPS values and the third range of IPS values include values very close together that may not be classified into two separate groups, for example, in a case of classifying an exemplary set of IPS values associated with a plurality of masses in a respective plurality of thyroid glands. In an exemplary implementation, determining the reference IPS based on the classified set of IPS values may comprise determining the reference IPS equal to a maximum IPS value among IPS values of the cancerous/precancerous IPS range.

In an exemplary implementation referring to FIG. 3B, step 310 may comprise detecting the cancerous status of exemplary suspected mass 206 based on the calculated IPS. In an exemplary implementation, detecting the cancerous status of exemplary suspected mass 206 may comprise looking up the calculated IPS and the measured electrical impedance magnitude of exemplary suspected mass 206 at frequency of 1 kHz (Z_{1 kHz}) in the calibration dataset. In an exemplary implementation, detecting the cancerous status of exemplary suspected mass 206 may comprise detecting exemplary suspected mass 206 being a benign region if the measured Z_{1 kHz} is less than the reference impedance value of the calibration dataset and the calculated IPS is more than the reference IPS of the calibration dataset. In an exemplary implementation, detecting the cancerous status of exemplary suspected mass 206 may comprise detecting exemplary suspected mass 206 being an excision-requiring cancerous or precancerous mass if the measured Z_{1 kHz} is less than the reference impedance value of the calibration dataset and the calculated IPS is less than the reference IPS of the calibration dataset. In an exemplary implementation, detecting the cancerous status of exemplary suspected mass 206 may comprise detecting exemplary suspected mass 206 being a precancerous mass if the measured Z_{1 kHz} is less than the reference impedance value of the calibration dataset and the calculated IPS is within the second range of IPS values. In an exemplary implementation, detecting the cancerous status of exemplary suspected mass 206 may comprise detecting exemplary suspected mass 206 being a cancerous mass if the measured Z_{1 kHz} is less than the reference impedance value of the calibration dataset and the calculated IPS is within the third range of IPS values.

In an exemplary embodiment, an exemplary generated calibration dataset may comprises a plurality of calibration datasets generated for a plurality of respective tissues or organs in human body where a suspected mass to be cancerous or precancerous may be found. In an exemplary embodiment, an exemplary generated calibration dataset may depend on electrical properties of a respective tissue or organ. In an exemplary embodiment, an exemplary generated calibration dataset may comprise an exemplary reference impedance value of 2000Ω and an exemplary reference IPS value of 2 for a case of detecting the cancerous status of an exemplary suspected mass 206 located in a thyroid gland. In an exemplary embodiment, an exemplary generated calibration dataset may comprise an exemplary reference impedance value of 1500Ω and an exemplary reference IPS value of zero for a case of detecting the cancerous status of an exemplary suspected mass 206 located in a breast tissue.

In an exemplary embodiment, steps 304 to 310 of exemplary method 300 may be done for more than one location of exemplary suspected mass 206 in a case if exemplary suspected mass 206 is a large or heterogeneous suspected mass. In an exemplary embodiment, a small suspected mass may comprise a cube-shaped suspected mass with an exemplary volume of about 20 mm³. In an exemplary embodiment, a large suspected mass may be more extended within a tissue with higher dimensions with a volume of more than about 20 mm³.

In an exemplary embodiment, suspected mass 206 may comprise a suspected mass that may require excision due to be cancerous or precancerous that may be located everywhere in a living body of a human or an animal. In an exemplary embodiment, “suspected mass” may refer to a mass that is suspected to be a cancerous mass or a precancerous mass. In an exemplary embodiment, a mass is found to be cancerous or precancerous, it may require to be excised. In an exemplary embodiment, such a mass which may be required to be excised may be referred to as an “excision-required tumor or mass” or “excision-requiring tumor or mass”. In an exemplary embodiment, suspected mass 206 may comprise a suspected mass to be a precancerous tumor mass or high risk mass or a borderline mass that may form a cancerous tumor in the future, so that suspected mass 206 may be an excision-requiring mass.

In an exemplary embodiment, detecting the cancerous status of exemplary suspected mass 206 may comprise detecting the cancerous status of a suspected mass to be cancerous/precancerous located in a breast tissue. In an exemplary implementation, detecting the cancerous status of suspected mass 206 based on the calculated IPS may comprise detecting suspected mass 206 is a cancerous, precancerous, or a benign breast lesion. In an exemplary implementation, detecting the cancerous status of suspected mass 206 may comprise detecting suspected mass 206 is a benign (healthy) region in the breast tissue if the calculated IPS is within an exemplary first range of IPS values comprising IPS values of more than zero. In an exemplary implementation, detecting the cancerous status of suspected mass 206 may comprise detecting suspected mass 206 is a cancerous (malignant) breast lesion if the calculated IPS is within an exemplary third range of IPS values comprising IPS values of less than an IPS value of −2. In another exemplary implementation, detecting the cancerous status of suspected mass 206 may comprise detecting suspected mass 206 is a precancerous breast lesion (a borderline breast lesion or a high-risk breast lesion) if the calculated IPS is within an exemplary second range of IPS values comprising IPS values in a range between −2 and 0. In an exemplary implementation, exemplary method 300 may further comprise a step 312 (not illustrated) for a case of status detection of a suspected mass 206 located in a breast tissue. In an exemplary implementation, step 312 may comprise detecting suspected mass 206 may be a non-excision-required benign mass in a breast tissue, where suspected mass 206 may be at least one of a fat necrotic region, a benign glandular region, and combinations thereof if the measured Z_{1 kHz} is more than the reference impedance value (1500Ω).

In an exemplary embodiment, exemplary electrical probe 100 and system 200 may be utilized via exemplary method 300 to conduct a real-time bioelectrical diagnostic mechanism for early detection of a cancerous tumor or a precancerous mass that may require excision before a cancerous states progresses further. In an exemplary embodiment, exemplary method 300 may be carried out, utilizing exemplary electrical probe 100 and system 200, as a complementary diagnostic process for improving breast imaging-reporting and data system (BI-RADS) scoring accuracy, especially in high-risk or borderline breast diseases for early detection of high-risk lesions and to help to increase an accuracy of tumor sampling and pathological diagnosis. In an exemplary embodiment, by precise calibration of IPS for suspected masses in an exemplary breast tissue, borderline breast diseases and high-risk lesions (such as papillary lesions, complex sclerosing adenosis, and fibroadenoma, extensive area of columnar cell changes, atypical ductal hyperplasia, etc.) that should be dissected according to standard guidelines can be diagnosed highly-sensitive (about 95.6%) before any sampling process. It is a precious outcome for high-risk lesions that may be radiologically underestimated to BI-RADS3, specifically in younger patients with dense breast masses that may be challenging in mammographic and even sonographic evaluations. Also, as many diagnostic errors may be related to imprecise samplings, the lowest IPS value may be associated to the most pathologic portions of a tumor for increasing sampling accuracy, which may be a valuable outcome for interventional radiologists, especially in more extensive tumors. In an exemplary embodiment, precise detection of high-risk breast masses, which may be declared as BI-RADS3 instead of BI-RADS4a may be achieved utilizing exemplary method 300.

In an exemplary embodiment, detecting the cancerous status of suspected mass 206 based on the calculated IPS may comprise detecting the cancerous status of a suspected nodule to be cancerous or precancerous which may be located in a thyroid gland. In an exemplary implementation, detecting the cancerous status of suspected mass 206 may comprise detecting the suspected nodule in the thyroid gland being one of a cancerous thyroid nodule and a precancerous (high risk) thyroid nodule if the calculated IPS is less than a reference IPS value of 2. In an exemplary implementation, detecting the cancerous status of suspected mass 206 may comprise detecting the suspected nodule in the thyroid gland being a benign thyroid nodule if the calculated IPS is more than the reference IPS value of 2.

In an exemplary implementation, steps 306 to 310 of exemplary method 300 may be carried out in less than one minute, including detecting an excision-requiring status of suspected mass 206 based on determining a cancerous status of suspected mass 206 or a possibility of transforming suspected mass 206 to a cancerous tumor in future. In an exemplary implementation, steps 306 to 310 of exemplary method 300 may be carried out by processing unit 204 utilizing electrical probe 100 and impedance analyzer device 202. In an exemplary implementation, processing unit 204 may include a memory having processor-readable instructions stored therein and a processor. The processor may be configured to access the memory and execute the processor-readable instructions.

In an exemplary implementation, the processor may be configured to perform a method by executing the processor-readable instructions. In an exemplary implementation, the method may include conducting steps 306 to 310 of exemplary method 300 utilizing impedance analyzer device 202 and electrical probe 100. In an exemplary implementation, the method may include applying an alternating current (AC) voltage in a sweeping range of frequencies to two electrodes 102 and 104 of electrical probe 100 being in contact with suspected mass 206, measuring an electrical impedance magnitude of suspected mass 206 at a frequency of 1 kHz (Z_{1 kHz}), plotting an impedance phase diagram by measuring a set of electrical impedance phase values from suspected mass 206 respective to the swept range of frequencies, calculating an impedance phase slope (IPS) of the plotted impedance phase diagram in a frequency range between 100 kHz and 500 kHz responsive to the measured Z_{1 kHz} being less than a reference impedance value, and detecting cancerous status of suspected mass 206 based on the calculated IPS. In an exemplary implementation, the processor may be further configured to record and communicate the measured set of electrical impedance phase values, the calculated IPS, and the measured Z_{1 kHz} from suspected mass 206 to an individual or an expert who may utilize processing unit 204.

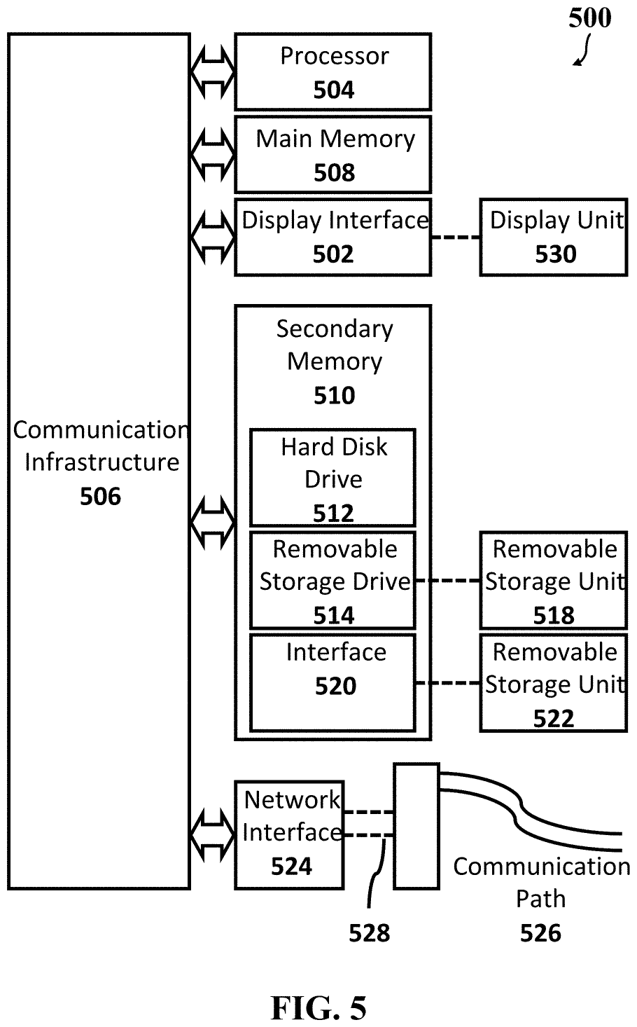

FIG. 5 shows an example computer system 500 in which an embodiment of the present disclosure, or portions thereof, may be implemented as computer-readable code, consistent with one or more exemplary embodiments of the present disclosure. For example, computer system 500 may include an example of processing unit 204, steps 306, 308, and 310 of exemplary flowchart 300 presented in FIGS. 3A and 3B, and steps 422, 424, and 426 of exemplary flowchart 400 presented in FIG. 4B may be implemented in computer system 500 using hardware, software, firmware, tangible computer readable media having instructions stored thereon, or a combination thereof and may be implemented in one or more computer systems or other processing systems. Hardware, software, or any combination of such may embody any of the modules and components in FIG. 2, FIGS. 3A and 3B, and FIG. 4B.

If programmable logic is used, such logic may execute on a commercially available processing platform or a special purpose device. One ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores.”

An embodiment of the present disclosure is described in terms of this example computer system 500. After reading this description, it will become apparent to a person skilled in the relevant art how to implement the invention using other computer systems and/or computer architectures. Although operations may be described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

Processor device 504 may be a special purpose or a general-purpose processor device. As will be appreciated by persons skilled in the relevant art, processor device 504 may also be a single processor in a multi-core/multiprocessor system, such system operating alone, or in a cluster of computing devices operating in a cluster or server farm. Processor device 504 may be connected to a communication infrastructure 506, for example, a bus, message queue, network, or multi-core message-passing scheme.

In an exemplary embodiment, computer system 500 may include a display interface 502, for example a video connector, to transfer data to a display unit 530, for example, a monitor. Computer system 500 may also include a main memory 508, for example, random access memory (RAM), and may also include a secondary memory 510. Secondary memory 510 may include, for example, a hard disk drive 512, and a removable storage drive 514. Removable storage drive 514 may include a floppy disk drive, a magnetic tape drive, an optical disk drive, a flash memory, or the like. Removable storage drive 514 may read from and/or write to a removable storage unit 518 in a well-known manner. Removable storage unit 518 may include a floppy disk, a magnetic tape, an optical disk, etc., which may be read by and written to by removable storage drive 514. As will be appreciated by persons skilled in the relevant art, removable storage unit 518 may include a computer usable storage medium having stored therein computer software and/or data.

In alternative implementations, secondary memory 510 may include other similar means for allowing computer programs or other instructions to be loaded into computer system 500. Such means may include, for example, a removable storage unit 522 and an interface 520. Examples of such means may include a program cartridge and cartridge interface (such as that found in video game devices), a removable memory chip (such as an EPROM, or PROM) and associated socket, and other removable storage units 522 and interfaces 520 which allow software and data to be transferred from removable storage unit 522 to computer system 500.

Computer system 500 may also include a communications interface 524. Communications interface 524 allows software and data to be transferred between computer system 500 and external devices. Communications interface 524 may include a modem, a network interface (such as an Ethernet card), a communications port, a PCMCIA slot and card, or the like. Software and data transferred via communications interface 524 may be in the form of signals, which may be electronic, electromagnetic, optical, or other signals capable of being received by communications interface 524. These signals may be provided to communications interface 524 via a communications path 526. Communications path 526 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 518, removable storage unit 522, and a hard disk installed in hard disk drive 512. Computer program medium and computer usable medium may also refer to memories, such as main memory 508 and secondary memory 510, which may be memory semiconductors (e.g. DRAMs, etc.).

Computer programs (also called computer control logic) are stored in main memory 508 and/or secondary memory 510. Computer programs may also be received via communications interface 524. Such computer programs, when executed, enable computer system 500 to implement different embodiments of the present disclosure as discussed herein. In particular, the computer programs, when executed, enable processor device 504 to implement the processes of the present disclosure, such as the operations in method 300 illustrated by FIGS. 3A and 3B, or method 420 illustrated by FIG. 4B discussed above. Accordingly, such computer programs represent controllers of computer system 500. Where an exemplary embodiment of methods 300 or 420 is implemented using software, the software may be stored in a computer program product and loaded into computer system 500 using removable storage drive 514, interface 520, and hard disk drive 512, or communications interface 524.

Embodiments of the present disclosure also may be directed to computer program products including software stored on any computer useable medium. Such software, when executed in one or more data processing device, causes a data processing device to operate as described herein. An embodiment of the present disclosure may employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

### Example 1: Fabrication of a Single-Needle Electrical Probe with Two Integrated Needle Electrodes

In this example, an exemplary probe similar to probe 100 was designed and fabricated. Exemplary fabricated probe includes two standard medical-grade needles utilized as exemplary electrodes 104 and 102. A 20 G×90 mm disposable spinal cannula was used as an inner electrode similar to second electrode 104 and a 14 G standard I.V. cannula with an injection valve was used as an outer electrode similar to first electrode 102. The two standard medical-grade needles are electrically isolated and soldered to two miniaturized banana connectors similar to electrical connectors 118 and 120. Each banana connector has a cylindrical metal head with a diameter of about 2 mm. An exemplary plastic shield covers the outer needle. A distal end portion of the inner electrode with a length of about 2 mm and a distal end portion of the outer electrode with a length of about 5 mm are uncovered similar to distal end portions 116 and 112 of exemplary electrodes 104 and 102. The uncovered distal ends are configured to be exposed to an exemplary suspected mass 206. An exemplary distance between two exemplary distal end portions of the inner and outer electrodes is about 5 mm. A thickness of a plastic insulator between inner and outer electrodes may be maximized to prevent liquid substances such as blood, extracellular fluid, lactations, or infectious liquids flow through it, hence decreasing efficiency of exemplary fabricated probe in impedance spectroscopy. Two needle electrodes of exemplary fabricated probe may be utilized as conductive electrodes to apply alternating voltage as stimulation and pass an electrical current through a body tissue as a response. Exemplary inner electrode is isolated from exemplary outer electrode and can move through it. In an exemplary implementation of exemplary method 300 described herein above, in step of putting the inner and outer electrodes in contact with exemplary suspected mass 206, first, exemplary 14 G cannula is inserted into suspected mass 206 with Ultra Sonography (US) guidance. Then, exemplary 20 G needle is pressed to a place within suspected mass 206 in a target position for conducting impedance spectroscopy.

### Example 2: Electrical Impedance Measurement and Safety Evaluations in the Mouse Model

In this example, before human tests, an exemplary probe similar to electrical probe 100 was utilized via an exemplary method and system similar to method 300 and system 200 for investigating detection of excision-required masses due to their cancerous status in healthy and tumoral tissues of 5 mice models under ketamine (50 mg/kg) and xylazine anesthesia (10 mg/kg) in an in-vivo and real-time approach.

In detail, 4T1 cell lines (a mouse type breast cancer cell line with invasive phenotypes) were maintained in Dulbecco's Modified Eagle Medium (DMEM) culture medium complimented with about 5% fetal bovine serum and about 1% penicillin/streptomycin at 37° C. with about 5% flow of CO₂ and about 95% filtered air. Primary populations of the cultured cell lines were determined manually by Haemocytometer Neubauer.

A total of about 2×10⁶ 4T1 cells per about 0.2 ml were implanted subcutaneously into back of 10 female BALB/C mice (5-6 weeks), under ketamine and xylazine anesthesia, 50 mg/kg and 10 mg/kg i.p., respectively. After 14 days, an exemplary method similar to method 300 utilizing an exemplary probe similar to electrical probe 100 was applied to five tumors and five normal tissues of the mice with the same sizes of formed tumors and sharp histological distinct patterns. Maximum passing electrical current (and hence transmitted power) during impedance spectroscopy was measured to be ensured about electrical safety of the utilized probe and method for mice' body and also to have a pre-estimation from electrical impedance responses of normal and cancerous tissues of mice.

Impedance spectroscopy results on a cohort of mice model (5 female BALB/C mice each had been subcutaneously tumorized by 4×10⁶ 4T1 cells as triple-negative mouse breast cancer cell-lines) showed a maximum electric current of about 800 μA through a portion of tissue confined between two needle electrodes of exemplary probe (at a voltage of about 0.8 V) and transmitted power of about 0.16 mW, which are entirely in safe mode.

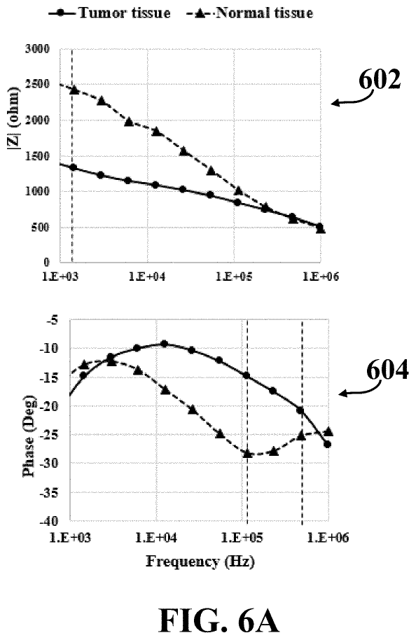

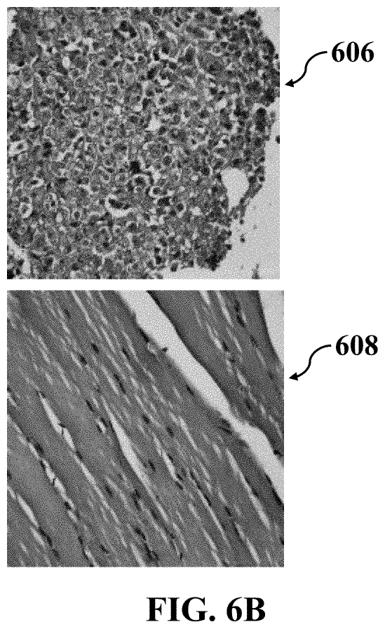

FIG. 6A shows comparison of frequency responses of muscular (normal) and cancerous tissues of an exemplary mouse model, consistent with one or more exemplary embodiments of the present disclosure. Responses shown in up panel 602 represents impedance magnitude for normal and cancerous tissue and responses shown in bottom panel 604 represents impedance phase diagram for normal and cancerous tissue. Drastic differences in impedance phase variation regime were observed between responses of healthy muscle and cancerous lesions. Comparing frequency response of muscular and tumor tissue of mice model shows a drastic difference in slope of phase diagram in a frequency range of 100 kHz to 500 kHz (called IPS). IPS is the most critical parameter in classifying tissues utilizing exemplary method 300 and system 200 described herein above. Similar to results obtained from test on exemplary suspected masses in breast tissue of human patients in following example, a calculated IPS value is a positive value for healthy muscular tissue of mice's breast tissue while it is a negative value for cancerous breast regions in mice models. As can be inferred from FIG. 6A, an exemplary normal muscular tissue of the mouse model shows a positive IPS while an exemplary cancerous tissue shows a sharp negative IPS. Besides, FIG. 6B shows comparative histopathological H&E assays of an exemplary healthy tissue and an exemplary cancerous tissue an exemplary mouse model of FIG. 6A, consistent with one or more exemplary embodiments of the present disclosure. As illustrated in FIG. 6B, muscular tissue mimics a fibrotic structure with a low and small cellular structure (image 608), while cancerous tissue represents a hypercellular region with irregularly large and deformed neoplastic cells showing vesicular nuclei (image 606).

### Example 3: Detection of Excision Recommended Tumors in Human Patients

In this example, an exemplary fabricated probe (described in EXAMPLE 1 hereinabove) via an exemplary system similar to system 200 was utilized for conducting a process similar to method 300 for detecting excision-requiring cancerous, precancerous, or high-risk masses in human bodies. This example includes an in-vivo distinction of high-risk breast lesions for 138 patients with respective 138 suspected masses similar to suspected mass 206 in their breast tissue. In-vivo distinction of high-risk breast lesions was done based on measuring and calculating an IPS value according to exemplary method 300 before any pathological diagnosis or BI-RADS scoring.

For minimizing intervention purposes, only patients with a surgeon's request for biopsy according to general guidelines were included in tests. BI-RADS scores were given based on mammography/Ultra Sonography (US) in patients older than 40 years and only US for patients younger than 40. Due to the unknown effects of blood existence on impedance measurements, seven patients with bleeding during tests were excluded. Blood has a high dielectric constant and may affect impedance measurement. This effect is compensated by thickening an exemplary plastic insulator between exemplary two electrodes of an exemplary fabricated and utilized probe. Therefore, 138 patients including 123 patients with BI-RADS3 and 15 patients with BI-RADS of BI-RADS4a or larger who were candidates for Core Needle Biopsy (CNB) (n=128), Vacuum-Assisted Biopsy (VAB) (n=9), and Fine Needle Aspiration (FNA) (n=1) were included in model test cohort herein. Fifteen BI-RADS3 patients had a surgeon's request for biopsy because of clinical indications. All cases were studied by two pathologists independently, and in case of disagreement, a final consensus was established. Exemplary two electrodes of exemplary fabricated probe were inserted into 138 human palpable breast lesions undergo CNB, VAB, or FNA with surgeon's requests. Impedance phase slope in frequency ranges of 100 kHz to 500 kHz (IPS) was extracted as exemplary classification parameter. Consistency of radiological and pathological declarations for excisional recommendation was then compared with the IPS values. Considering pathological results as gold standard, meaningful correlations between IPS and pathological status of lesions recommended for excision observed (p<0.0001). IPS was associated with the benign, high-risk proliferative, precancerous, or cancerous status of an exemplary lesion based on breast tissue's dielectric properties.

A customized precision impedance meter that works in constant voltage mode was used as an exemplary device similar to impedance analyzer device 202. An alternative signal with a constant voltage of about 0.4 V amplitude and frequency range of 1 Hz to 500 kHz is applied between exemplary inner and outer needles of exemplary fabricated probe. The applied voltage may stimulate an exemplary tissue with an electric field of about 200 V/m. An electrical current limiter adjusted in approximately 1 mA may also be used to control an electric current passing through an exemplary tissue for safety purposes. A magnitude of about 5 mA may be the maximum current that a human body can pass without any electrical shock. Impedance magnitude and phase diagrams may then be calculated, and IPS in a frequency range of 100 kHz to 500 kHz and Z_{1 kHz} (impedance magnitude in frequency 1 kHz) may be extracted as classification parameters. Finally, parameters calibration was performed by examination of 138 breast tumors of 138 eligible patients tested herein who were tested also with a biopsy from a suspected mass similar to suspected mass 206.

In a sonography room, candidates were tested via exemplary method 300 after local anesthesia (two-step Lidocaine injection for CNB and Lidocaine/Epinephrine injection for VAB) under US guidance. First, US may confirm that exemplary electrodes may be carefully inserted into exemplary suspected mass 206. Then, targeted US was performed using a 7 to 12 MHz multi-frequency probe. At least three points of each exemplary suspected mass 206 of each of 138 patients were tested with a measurement time of about 10 seconds for each point. Extracted parameters from each tested exemplary suspected mass 206 were then compared with pathological diagnostics of an exemplary respective biopsied sample from each of 138 patients.

Permanent histopathology of biopsied samples was an exemplary gold standard for diagnostic score of suspected masses, and both sonography and impedance-based results were compared to this standard. All cases were studied by two pathologists independently, and in a case of disagreement, a final consensus was established. Patients were biopsied just due to a surgeon's request through a radiologist's recommendation or clinical examinations. Table 1 shows basic information about tests population. Exemplary cohort study included benign (BI-RADS3, n=15), suspicious (BI-RADS4, n=101), and highly-suspicious (BI-RADS5, n=22) breast masses in which malignancies occurred in 28.3% (39/138) of the population. In comparison, benign and borderline breast lesions include 33.3% (46/138) and 38.4% (53/138) of the patients, respectively, based on radiological and pathological evaluations. Exemplary tests were initialized by examining radiologically BI-RADS5 (highly suspicious) and BI-RADS4 (suspicious) lesions (an observational study). After obtaining primary calibrations, the tests were developed to carry out intervention on probably benign cases (BI-RADS3). Observational studies of IPS and pathological evaluations showed meaningful relations between the high-risk proliferative, precancerous, or cancerous status of the lesions and their dielectric properties.

TABLE 1
Basic information of patients in exemplary study cohort (“Diameter”
refers to the largest diameter of an exemplary lesion).

| | All patients (n = 138) | | | |
|---|---|---|---|---|
| | Benign | Borderline | Malignant | |
| | (n = 46) | (n = 53) | (n = 39) | P-value |
| Age (years) | 35.5 ± 9.8 | 36.3 ± 9.1 | 49.5 ± 8.8 | 0.001 |
| >50 | 2 (4.35%) | 2 (3.8%) | 14 (35.9%) | |
| 35 to 50 | 19 (41.3%) | 27 (50.9%) | 23 (59%) | |
| ≤35 | 25 (54.35%) | 24 (45.3%) | 2 (5.1%) | |
| Diameter (mm) | 25.8 ± 21.8 | 28.4 ± 17.8 | 29.4 ± 18.5 | 0.076 |
| >30 | 10 (21.7%) | 18 (34%) | 12 (30.8%) | |
| >20, ≤30 | 14 (30.4%) | 15 (28.3%) | 12 (30.8%) | |
| >10, ≤20 | 13 (28.3%) | 13 (24.5%) | 12 (30.8%) | |
| ≤10 | 9 (20.6%) | 7 (13.2%) | 3 (7.6%) | |
| BI-RADS | | | | 0.00002 |
| 3 | 9 (19.6%) | 6 (11.3%) | 0 (0%) | |
| 4a | 32 (69.5%) | 42 (79.2%) | 1 (2.5%) | |
| 4b | 4 (8.7%) | 5 (9.5%) | 3 (7.7%) | |
| 4c | 1 (2.2%) | 0 (0%) | 13 (33.3%) | |
| 5 | 0 (0%) | 0 (0%) | 22 (56.5%) | |
| IPS | 1.1 ± 2.1 | −1.9 ± 1.5 | −3.5 ± 1.6 | 1.4E−15 |
| >0 | 40 (87%) | 3 (5.7%) | 1 (2.6%) | |
| −2 to 0 | 2 (4.35%) | 31 (58.5%) | 2 (5.1%) | |
| <−2 | 4 (8.7%) | 19 (35.8%) | 36 (92.3%) | |

Based on measured IPS values, breast tumor lesions were classified into two groups, including masses required for excision or treatment due to their malignancy probability and lesions not required for excision (or treatment). Positive score based on IPS value may be equal to an evidence that the scored mass must be excised or treated (which may need either direct excision or post radio/chemotherapy excision). So, intra-radiological biopsy would be suggested for these cases to better evaluate nature of an exemplary suspicious mass.

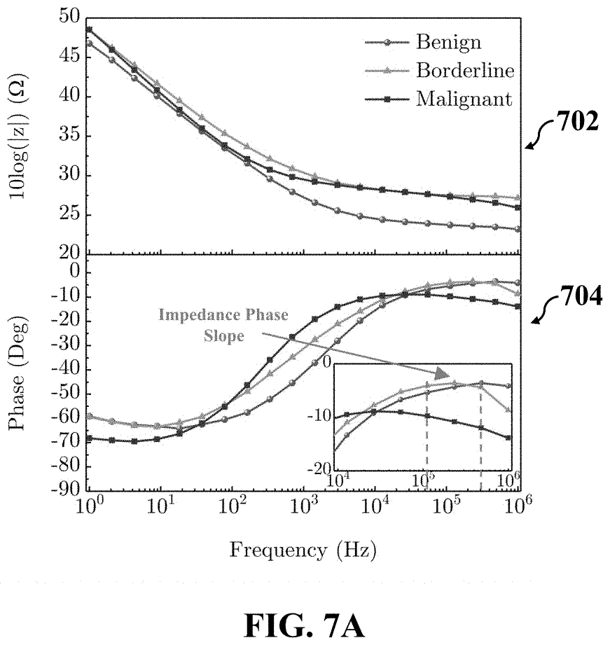

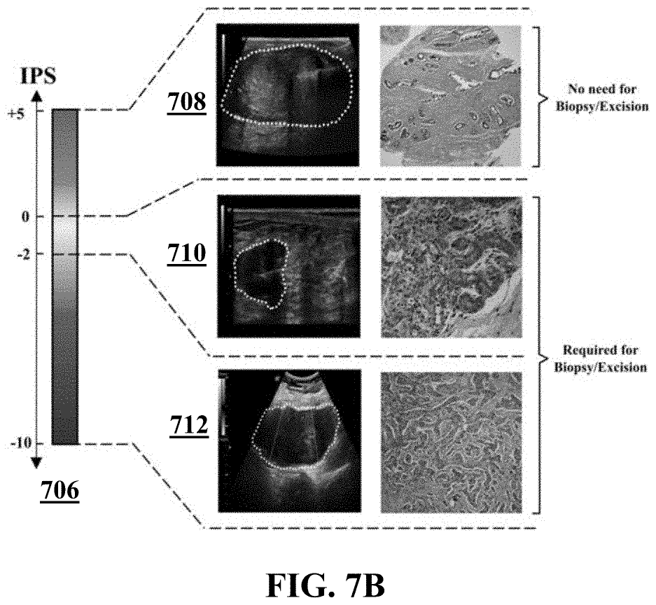

FIGS. 7A and 7B show classification of breast tumor masses into two classes based on calculated IPS values for a lesion that is not required for biopsy/surgery and lesions required for biopsy/surgery. FIG. 7A shows impedance magnitude (diagram 702) and phase diagram 704 for benign, high-risk benign (borderline), and malignant breast lesions, consistent with one or more exemplary embodiments of the present disclosure. IPS is a positive value for lesions that are not required for biopsy/surgery, while it is negative for two subgroups of tumors required for biopsy/surgery.

FIG. 7B shows an exemplary scale bar 706 for breast lesion classification with IPS and US images and H&E assays for a low-risk benign lesion BI-RADS4a (part 708), a borderline breast disease (complex sclerosing adenosis) BI-RADS4a (part 710), and a malignant lesion with BI-RADS5 (part 712), consistent with one or more exemplary embodiments of the present disclosure. According to exemplary scale bar 706, considering zero as an IPS cut-off value, breast lesions may be scored positive or negative for biopsy/surgery. Regarding part 708, US images and H&E assay (100×) of a low-risk benign lesion BI-RADS4a, CNB result scored it negative for malignancy. It also scored negative for biopsy/surgery by exemplary method 300. US images and H&E assay (100×) of borderline breast disease (complex sclerosing adenosis), BI-RADS4a are illustrated in part 710. It was scored positive for biopsy/surgery by exemplary method 300 by obtaining a negative IPS value between −2 and 0. US images and H&E assay (100×) of an exemplary malignant lesion are illustrated in part 712. Exemplary US image shows a BI-RADS5 lesion with hyper-vascularity and infiltrative borders. It was scored positive for biopsy/surgery by exemplary method 300, and a negative IPS value below −2 was obtained.

As illustrated in FIGS. 7A and 7B, after pathological evaluation and matching data with obtained results via exemplary method 300, it was observed that benign or low-risk breast tissues such as simple fibroadenoma (FA), simple sclerosing adenosis (SA), fibrocystic changes (FCC), usual ductal hyperplasia (UDH), etc. showed positive IPS values in impedance spectroscopy tests. These lesions are not required for biopsy/excision, and it is expected to be scored as BI-RADS3 during a sonography diagnostic procedure. Sonographic analysis of a benign solid mass (scored as BI-RADS3 and had positive IPS) shows a circumscribed and well-defined margin, oval shape, with minimum vascularity and minimum lobulated structure. Short-interval (6-month) follow-up sonography and then periodic sonographic surveillance may represent appropriate management. According to Error! Reference source not found., 7/15(47%) of lesions scored BI-RADS3 by the radiologist classified positive for biopsy/excision with exemplary method 300 (i.e., IPS<0). Pathological evaluations confirmed the existence of BBD in 6 patients (40%). As these lesions are recommended for 3 to 6 months follow-ups and hence late diagnosis, exemplary method 300 can effectively guide a radiologist or surgeon to biopsy tumors with BI-RADS3 or not. Thus, false negative of such radiological scoring would be reduced and helps an early diagnosis of patients. This precise diagnosis is especially vital for patients with a family history of breast cancer and young women of gestational age planning to become pregnant.

On the other hand, radiological evaluation scored low-risk benign lesions as BI-RADS3 (19.6%), BI-RADS4a (69.6%), BI-RADS4b (8.7%), and BI-RADS4c (2.1%). So, there were about 81.6% unnecessary biopsies in low-risk benign lesions, while applying exemplary method 300 only 18.4% unnecessary biopsies was recommended. Thus, as illustrated in Error! Reference source not found., about 81.6% of low-risk benign breast lesions represented positive IPS values (negative score) that support an idea that biopsy or surgical excision is not recommended for these lesions.

Two types of lesions are crucial to be genuinely recommended for biopsy by radiologists. Without biopsy and pathological investigations, some of these masses may be missed for therapeutic purposes.

First, high-risk proliferative breast lesions such as preneoplastic ductal/lobular lesions (ADH, ALH, and FEA), columnar cell hyperplasia (CCH) and extensive columnar cell changes alterations, benign fibroepithelial lesions (FEL) that resemble benign phyllodes tumors (such as hyper-cellular stroma, leaf-like structures with infiltrating attenuating layer), fibroadenomas with at least one criteria of complex FA (including SA, apocrine metaplasia, and cysts larger than 3 mm) or cellular FAs in old patients or patients with large tumor sizes and high growth rate tumors, papillary lesions of the breast, intraductal papilloma with or without atypia, radial scar, complex sclerosing adenosis, and extensive area of sclerosing adenosis. These lesions are recommended for surgical excision, but margin excision is not required. They may be miss-scored as BI-RADS3 in sonographic evaluation, and exemplary method 300 would be so helpful in discrimination of them from low-risk benign lesions. Interestingly, 94.3% of these lesions scored positive for excision by exemplary method 300, i.e., showed negative IPS (FIG. 7B, part 710).

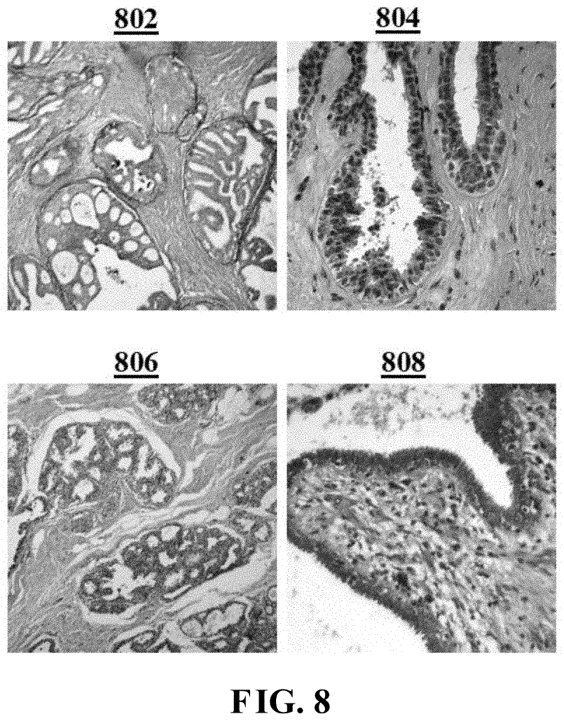

21 lesions from 90 BI-RADS3 or BI-RADS4a lesions was positively scored by exemplary method 300 (i.e., negative IPS), while their primary pathology results were negative and free from high-risk lesions. In an additional pathological study, all of these samples were examined by more microscopy re-checking, serial H&E sections, or IHC assays. These further evaluations corrected results to BBD and confirmed obtained result by exemplary method 300 in 14/90 (about 15.5%) of these samples. So, exemplary method 300 also showed a practical use as a complementary parameter for the pathologist to do more pathological investigations in suspicious samples. Picture of H&E assays of some of high-risk or borderline breast masses that had been scored positive by exemplary method 300 (negative IPS values) and recommended for excision are presented in FIG. 8. FIG. 8 shows histological patterns of H&E assay for patient ID87 with BI-RADS: B4a and IPS=−2.2 (malignant), CNB result: papillary lesion with ADH, Permanent pathology: DCIS, 100× (image 802), patient ID 95 with BI-RADS: B4a, IPS=−1.7 (borderline), CNB result: FEL in favor of FA, Permanent pathology: FA, moderate typical ductal hyperplasia, extensive area of CCC and CCH, 400× (image 804), patient ID49 with BI-RADS: B4a, IPS=−0.7 (borderline), CNB result: proliferative FCC, Permanent pathology: Sclerosing papillary lesion with lactational changes, complex SA, FCC, UDH, and simple adenosis, 100× (image 806), and patient ID63 with BI-RADS: B4a, IPS=−2.7, CNB result: FEL in favor of benign phyllodes tumor, Permanent pathology: Cellular FA, 100× (image 808), consistent with one or more exemplary embodiments of the present disclosure.

Next category of lesions that must be excised and treated are in-situ and invasive carcinoma components which show hyper-vascularity and infiltrative borders in sonographic evaluations. These lesions that are usually scored BI-RADS4b, BI-RADS4c, and BI-RADS5, confirmed negative IPS values through applying exemplary method 300 (FIG. 7B, part 712). As depicted in Error! Reference source not found., radiological evaluations of malignant tumors were found to be BI-RADS4a (2.6%), BI-RADS4b (7.7%), BI-RADS4c (33.3%), and BI-RADS5 (56.4%) in exemplary cohort study. As represented in FIG. 7B, part 712, malignant lesions showed negative IPS (92.3% of them had IPS less than −2) and were scored positive by exemplary method 300. It should be noted that low-grade carcinoma in situ has a low sensitivity on mammography and on ultrasound, too.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1- A method for real-time and in-vivo detecting cancerous status of a suspected mass to be cancerous/precancerous in a living body, comprising:
putting two electrodes of an electrical probe in contact with the suspected mass, the two electrodes connected to an impedance analyzer device;
recording an electrical impedance spectroscopy (EIS) from the suspected mass utilizing the impedance analyzer device, comprising:
applying an alternating current (AC) voltage in a sweeping range of frequencies to the two electrodes of the electrical probe;
measuring an electrical impedance magnitude of the suspected mass at a frequency of 1 kHz (Z_{1 kHz}); and
plotting an impedance phase diagram by measuring a set of electrical impedance phase values from the suspected mass respective to the swept range of frequencies;
calculating, utilizing one or more processors, an impedance phase slope (IPS) of the plotted impedance phase diagram in a frequency range between 100 kHz and 500 kHz responsive to the measured Z_{1 kHz} being less than a reference impedance value; and
detecting, utilizing one or more processors, cancerous status of the suspected mass based on the calculated IPS, comprising:
detecting the suspected mass being at least one of a cancerous mass and a precancerous mass responsive to the calculated IPS being less than a reference IPS; or
detecting the suspected mass being a benign region responsive to the calculated IPS being more than the reference IPS.

2- The method of claim 1, wherein calculating the IPS of the plotted impedance phase diagram in the frequency range between 100 kHz and 500 kHz comprises calculating the IPS from a relation defined by:
$$IPS = \frac{{Phase}_{2} - {Phase}_{1}}{log ( {Frequency}_{2} ) - log ( {Frequency}_{1} )} ,$$
wherein Phase₁ is a first measured impedance phase value at a first frequency value (Frequency₁) of 100 kHz and Phase₂ is a second measured impedance phase value at a second frequency value (Frequency₂) of 500 kHz.

3- The method of claim 2, further comprising generating a calibration dataset, comprising:
generating the reference impedance value, comprising:
measuring a set of Z_{1 kHz} values from a plurality of masses respective to a plurality of persons; and
determining the reference impedance value equal to a maximum value of the measured set of Z_{1 kHz} values; and
generating the reference IPS, comprising:
calculating, utilizing one or more processors, a set of IPS values from the plurality of masses;
determining cancerous status of each mass of the plurality of masses by applying a pathological assay to each mass, the determined cancerous status comprising one of a benign state, a precancerous state, and a cancerous state, based on result of the applied pathological assay;
assigning the determined status of each mass to the respective calculated IPS value;
classifying the calculated set of IPS values into three IPS ranges based on the determined cancerous status of each mass of the plurality of masses, the three IPS ranges comprising:
a benign IPS range comprising a first range of IPS values assigned as being of the benign state;
a precancerous IPS range comprising a second range of IPS values assigned as being of the precancerous state; and
a cancerous IPS range comprising a third range of IPS values assigned as being of the cancerous state; and
determining the reference IPS equal to a value between a minimum IPS value among IPS values of the benign IPS range and a maximum IPS value among IPS values of both the precancerous IPS range and the cancerous IPS range.

4- The method of claim 3, wherein:
putting two electrodes of the electrical probe in contact with the suspected mass comprises putting two electrodes of the electrical probe in contact with a suspected mass to be cancerous located in a breast tissue, and
detecting cancerous status of the suspected mass comprises detecting the suspected mass being at least one of a cancerous mass and a precancerous mass in the breast tissue responsive to the measured Z_{1 kHz} being less than the reference impedance value of 1500Ω and the calculated IPS being less than the reference IPS of zero.

5- The method of claim 4, wherein detecting the cancerous status of the suspected mass comprises:
detecting the suspected mass being a cancerous breast lesion responsive to the calculated IPS being less than an IPS value of −2; or
detecting the suspected mass being a precancerous breast lesion responsive to the calculated IPS being in a range of IPS value between −2 and 0.

6- The method of claim 3, wherein:
putting two electrodes of the electrical probe in contact with the suspected mass comprises putting two electrodes of the electrical probe in contact with a suspected nodule to be cancerous or precancerous located in a thyroid gland, and
detecting cancerous status of the suspected mass comprises detecting the suspected nodule being at least one of a cancerous thyroid nodule and a precancerous thyroid nodule responsive to the calculated IPS being less than the reference IPS value of 2 and the measured Z_{1 kHz} being less than the reference impedance value of 2000Ω.

7- The method of claim 1, wherein applying the AC voltage in the sweeping range of frequencies to the two electrodes of the electrical probe comprises:
connecting a first proximal end of a first electrode and a second proximal end of a second electrode of the electrical probe to the impedance analyzer device; and
applying an AC voltage in a sweeping range of frequencies between 1 kHz and 500 kHz to the two electrodes utilizing the impedance analyzer device.

8- The method of claim 1, wherein putting the two electrodes of the electrical probe in contact with the suspected mass comprises putting a first distal end of a first electrode and a second distal end of a second electrode of the electrical probe in contact with the suspected mass by inserting the first distal end of the first electrode and the second distal end of the second electrode inside the suspected mass.

9- The method of claim 8, wherein inserting the first distal end of the first electrode and the second distal end of the second electrode of the electrical probe inside the suspected mass comprises:
inserting a first distal end portion of the first electrode of the electrical probe through skin into the suspected mass, the first electrode comprising a first electrically conductive needle comprising a hollow needle; and
pushing a second distal end portion of the second electrode of the electrical probe through the first electrode into the suspected mass, the second electrode comprising a second electrically conductive needle placed inside the first electrode.

10- The method of claim 9, wherein the first electrically conductive needle comprises a stainless steel needle of at least one of a peripheral venous catheter and an intravenous (IV) cannula with a gauge size of 14 or more.

11- The method of claim 9, wherein the second electrically conductive needle comprises a stainless steel needle of a spinal cannula with a gauge size of 20 or more.

12- The method of claim 9, further comprising preparing the electrical probe, comprising:
coating a second electrically insulating layer on the second electrically conductive needle except a distal end portion of the second electrically conductive needle;
placing the second electrically conductive needle inside the first electrically conductive needle;
covering an outer surface of the first electrically conductive needle with a first electrically insulating layer except a distal end portion of the first electrically conductive needle; and
attaching two electrical connectors to the first electrically conductive needle and the second electrically conductive needle, comprising:
attaching a first electrical connector onto a surface of the first electrically conductive needle adjacent to a proximal end of the first electrically conductive needle; and
attaching a second electrical connector onto a proximal end of the second electrically conductive needle.

13- A system for real-time and in-vivo detecting cancerous status of a suspected mass to be cancerous/precancerous in a living body, comprising:
an electrical probe comprising:
a first electrode comprising a first electrically conductive needle, the first electrode comprising a first distal end portion and a first proximal end portion;
a first electrically insulating layer placed around the first electrode except the first distal end portion;
a second electrode comprising a second electrically conductive needle, the second electrode placed inside the first electrode, the second electrode movable in longitudinal direction along the first electrode, the second electrode comprising a second distal end portion and a second proximal end portion, the second distal end portion configured to be placed outside of the first electrode; and
a second electrically insulating layer placed between the first electrode and the second electrode,
wherein the first distal end portion and the second distal end portion are configured to be put in contact with the suspected mass;
an impedance analyzer device configured to be connected to the electrical probe; and
a processing unit electrically connected to the impedance analyzer device, the processing unit comprising:
a memory having processor-readable instructions stored therein; and
a processor configured to access the memory and execute the processor-readable instructions, which, when executed by the processor configures the processor to perform a method, the method comprising:
applying, utilizing the impedance analyzer device, an alternating current (AC) voltage in a sweeping range of frequencies to the first electrode and the second of the electrical probe;
measuring, utilizing the impedance analyzer device, an electrical impedance magnitude of the suspected mass at a frequency of 1 kHz (Z_{1 kHz});
plotting, utilizing the impedance analyzer device, an impedance phase diagram by measuring a set of electrical impedance phase values from the suspected mass respective to the swept range of frequencies;
calculating an impedance phase slope (IPS) of the plotted impedance phase diagram in a frequency range between 100 kHz and 500 kHz responsive to the measured Z_{1 kHz} being less than a reference impedance value; and
detecting cancerous status of the suspected mass based on the calculated IPS.

14- The system of claim 13, wherein calculating the IPS of the plotted impedance phase diagram in the frequency range between 100 kHz and 500 kHz comprises calculating the IPS from a relation defined by:
$$IPS = \frac{{Phase}_{2} - {Phase}_{1}}{log ( {Frequency}_{2} ) - log ( {Frequency}_{1} )} ,$$
Wherein Phase₁ is a first measured impedance phase value at a first frequency value (Frequency₁) of 100 kHz and Phase₂ is a second measured impedance phase value at a second frequency value (Frequency₂) of 500 kHz.

15- The system of claim 13, wherein detecting cancerous status of the suspected mass based on the calculated IPS comprises:
detecting the suspected mass being at least one of a cancerous mass and a precancerous mass responsive to the calculated IPS being less than a reference IPS; or
detecting the suspected mass being a benign region responsive to the calculated IPS being more than the reference IPS.

16- The system of claim 15, wherein the method further comprises generating a calibration dataset, comprising:
generating the reference impedance value, comprising:
measuring a set of Z_{1 kHz} values from a plurality of masses associated with a respective plurality of persons; and
determining the reference impedance value equal to a maximum value of the set of Z_{1 kHz} values; and
generating the reference IPS, comprising:
calculating, utilizing one or more processors, a set of IPS values from the plurality of masses;
determining cancerous status of each mass of the plurality of masses by applying a pathological assay to each mass, the determined cancerous status comprising one of a benign state, a cancerous state, and a precancerous state, based on result of the applied pathological assay;
assigning the determined status of each mass to the respective calculated IPS value;
classifying the set of IPS values into three IPS ranges based on the determined cancerous status of each mass of the plurality of masses, the three IPS ranges comprising:
a benign IPS range comprising a first range of IPS values assigned as being of the benign state;
a precancerous IPS range comprising a second range of IPS values assigned as being of the precancerous state; and
a cancerous IPS range comprising a third range of IPS values assigned as being of the cancerous state; and
determining the reference IPS equal to a value between a minimum IPS value among IPS values of the benign IPS range and a maximum IPS value among IPS values of both the precancerous IPS range and the cancerous IPS range.

17- The system of claim 16, wherein:
the suspected mass comprises a suspected mass to be cancerous or precancerous located in a breast tissue, and
detecting the cancerous status of the suspected mass comprises:
detecting the suspected mass being a cancerous breast lesion responsive to the calculated IPS being less than an IPS value of −2; or
detecting the suspected mass being a precancerous breast lesion responsive to the calculated IPS being in a range of IPS value between −2 and 0.

18- The system of claim 16, wherein:
the suspected mass comprises a suspected nodule to be cancerous or precancerous located in a thyroid gland,
the reference impedance value comprises an impedance value of 2000Ω, and
the reference IPS comprises an IPS value of 2.

19- The system of claim 18, wherein detecting cancerous status of the suspected mass comprises:
detecting the suspected nodule being one of a cancerous thyroid nodule and a precancerous thyroid nodule responsive to the calculated IPS being less than an IPS value of 2.
